SEQUENCE LISTING (53 sequences)

SEQ ID NO: 1 - DNA, 674 bp, Artificial Sequence
  pMB1 ori extracted from GenBank #EU496089
cccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 2 - DNA, 1554 bp, Artificial Sequence
  p15A ori extracted from GenBank #EU496097
atggaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgtt

SEQ ID NO: 3 - DNA, 2037 bp, Artificial Sequence
  pSC101 extracted from GenBank #EU496096
ctgtcagaccaagtttacgagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggacatgtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaac

SEQ ID NO: 4 - DNA, 943 bp, Artificial Sequence
  AmpR extracted from GenBank #EU496092
ctaaatacattcaaatatctatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgccgaagatcagttgggtgcacgtgtgggttacatcgaactggacctcaacagcggtaagattcttgagagttttcgccccgaagaacgtttcccaatgatgagcacttttaaagttctgctctgtggcgcggtattatcccgtattgacgccgggcaagagcaactcggtcgccgcatacactattctcagaatgacttggttgagtactcaccagtcacagaaaagcatcttacggacggcatgacagtacgcgaattatgcagcgctgccataaccatgagtgataacacggcggccaacttacttctgacaacgatcggaggaccgaaggagcttaccgcttttttgcacaacatgggtgatcatgtaactcgccttgatcgttgggaaccggagctgaatgaagccataccaaacgacgagcgtgacaccacgatgcctgtagctatggcaacaacgttgcgcaaactcttaactggcgaacttcttactctcgcttcccggcaacaattaatagactggatggaggcggataaagttgcaggaccacttctgcgctcggcccttccggctggctggtttattgctgataaatctggagccggtgagcgtgggtcccgcggtattattgcagccctggggccagatggtaagccctcccgtatcgtagttatctacacgacggggagccaggcaactatggacgaacgtaatcgccagatcgctgagataggtgcctccctgattaagcattggtaataa

SEQ ID NO: 5 - DNA, 769 bp, Artificial Sequence
  ChlorR extracted from GenBank #EU496094
gttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataa

SEQ ID NO: 6 - DNA, 967 bp, Artificial Sequence
  KanR extracted from GenBank #EU496093
ctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgtctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataa

SEQ ID NO: 7 - DNA, 1283 bp, Artificial Sequence
  TetR extracted from GenBank #EU496095
gagattctcatgtttgacagcttatcatcgataagctttaatgcggtagtttatcacagttaaattgctaacgcagtcaggcaccgtgtatgaaatctaacaatgcgctcatcgtcattctcggcaccgtcaccctggacgctgtaggcataggcttggttatgccggtactgccgggcctcttgcgggatatcgtccattccgacagtattgccagtcactatggcgtgctgcttgcgctctatgcgttgatgcaatttctttgcgcacccgttctcggagccctgtccgaccgctttggccgccgtccagtcctgctcgcttcgctccttggagccactatcgactacgcgatcatggcgaccacacccgtcctgtggattctctacgccggacgcatcgtggcgggcatcacgggtgccacaggtgcggttgctggtgcctatatcgccgacatcaccgatggggaagatcgggctcgccacttcgggctcatgagcgcttgtttcggcgtgggtatggtggcaggccccgtggccgggggactgttgggtgccatctccttgcatgcaccattccttgcggcggcggtgctcaacggcctcaacctcctcctgggctgcttccttatgcaggaatcgcataagggagagcgccgtccgatgcccttgcgtgccttcaatccagtcagctccttccggtgggcgcggggcatgactatcgtcgccgcacttatgactgttttctttatcatgcaactcgtaggacaggttccggcagcgctctgggtcattttcggcgaggaccgctttcgctggagcgcgacgatgatcggcctgtcgcttgcggtattcggaatcttgcacgccctcgctcaagccttcgtcacgggccccgccaccaaacgtttcggcgagaagcaggccattatcgcgggcatggcggccgacgcgctgggctacgtcttgctggcgttcgcgacgcgcggctggatggccttccccattatgattcttctcgcttccggcggcatcggtatgcccgcgttgcaggccatgctgtcccgccaagtagatgacgaccatcagggacagcttcaagggtcgctcgcggctcttaccagcctcacttcgatcattggaccgctgatcgtcacggcgatttatgccgcctcggcgagcacatggaacgggttggcatggattgtaggtgccgccctttaccttgtctgcctccccgcgttgcgtcgcggtgcatggagccgggccacctcgacctaataa

SEQ ID NO: 8 - DNA, 1080 bp, Artificial Sequence
  LacI, Part Bba_C0012, N and C terminal converted to wt version then placed in byte format.
tgggtccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcggggcct

SEQ ID NO: 9 - DNA, 711 bp, Artificial Sequence
  Lambda C1, Part BBa_C0051, C terminal sequence converted to wt version then placed in byte format.
tgggtacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttgcct

SEQ ID NO: 10 - DNA, 876 bp, Artificial Sequence
  AraC, Part BBa_C0080, C terminal sequence converted to wt version then placed in byte format.
tgggtgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttggcct

SEQ ID NO: 11 - DNA, 1080 bp, Artificial Sequence; identical to SEQ ID NO: 8
  TetRo, Part Bba_C0040, C terminal converted to wt version then placed in byte format.

SEQ ID NO: 12 - DNA, 772 bp, Artificial Sequence
  GFP, Part BBa_I13522
gaattcgcggccgcttctagaggtctcatgggtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagcctagagaccactagttgcggccgctgcag

SEQ ID NO: 13 - DNA, 680 bp, Artificial Sequence
  RFP, Part BBa_I13521
tgggttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataa

SEQ ID NO: 14 - DNA, 737 bp, Artificial Sequence
  CFP; BBa_K349113
ggtctcatgggtgtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggcctagagacc

SEQ ID NO: 15 - DNA, 982 bp, Artificial Sequence
  CrtB, Part K274100 (ind. K118002)
gaattcgcggccgcttctagaggtctcatgggtaatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccggcctagagaccactagttgcggccgctgcag

SEQ ID NO: 16 - DNA, 963 bp, Artificial Sequence
  CrtE, Part K274100 (ind. I742151)
gaattcgcggccgcttctagaggtctcatgggtgtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggcccgagggcggttgaagaacgtctgagacaacatcttcagcttgccagtgagcatctctctgcggcctgccaacacgggcacgccactcaacattttattcaggcctggtttgacaaaaaactcgctgccgtcaggcctagagaccactagttgcggccgctgcag

SEQ ID NO: 17 - DNA, 1531 bp, Artificial Sequence
  CrtI, Part K274100 (ind. K118003)
gaattcgcggccgcttctagaggtctcatgggtccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaagcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcaaactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctggcctagagaccactagttgcggccgctgcag

SEQ ID NO: 18 - DNA, 1201 bp, Artificial Sequence
  CrtY, Part K274200 (ind. K118008)
gaattcgcggccgcttctagaggtctcatgggtccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatgcctagagaccactagttgcggccgctgcag

SEQ ID NO: 19 - DNA, 583 bp, Artificial Sequence
  CrtZ, Part I742157
gaattcgcggccgcttctagaggtctcatgggttggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaaggcctagagaccactagttgcggccgctgcag

SEQ ID NO: 20 - DNA, 1312 bp, Artificial Sequence
  VioA, Part K274002
gaattcgcggccgcttctagaggtctcatgggtcattcttccgatatctgcattgttggtgctggtatttctggtttgacgtgcgcaagccatctgctggacagcccggcatgccgtggtctgagcctgcgtatctttgacatgcagcaagaagccggtggccgtatccgcagcaaaatgctggatggtaaggcaagcattgaactgggcgcaggtcgctactcccctcagttgcacccgcatttccaaagcgcaatgcagcactatagccaaaagagcgaagtctatccgttcacccagttgaagttcaaatctcacgtgcagcaaaagctgaagcgcgccatgaatgaactgtccccgcgtctgaaagagcatggtaaagagagctttttgcagtttgtcagccgttatcaaggtcacgatagcgcggttggtatgatccgctctatgggttacgacgcactgttcctgccggatatcagcgcagaaatggcctacgacattgtgggtaagcacccggagatccagagcgtgacggacaacgacgcgaaccaatggtttgcagcggaaacgggctttgctggtctgattcagggcatcaaggctaaggttaaggcggcaggtgcgcgttttagcctgggttatcgtctgctgagcgtccgtaccgacggtgacggctacctgctgcaactggcaggtgacgacggctggaaactggagcaccgtacccgccatctgattctggcgattccgccgagcgcgatggcgggtttgaatgttgattttccagaagcctggtccggtgcgcgctatggcagcctgccgctgtttaagggctttctgacgtacggtgagccgtggtggttggactacaaactggacgatcaggtgctgattgttgacaacccgctgcgcaaaatctatttcaaaggcgataagtacctgttcttctataccgatagcgagatggcgaattactggcgcggttgtgtcgcggagggcgaggacggttacctggagcaaattcgcacccatttggctagcgcactgggtatcgtccgtgaacgtatcccgcaaccgctggcacacgttcacaagtattgggcgcacggcgttgagttttgccgtgattctgatattgaccacccgagcgcactgtctcatcgcgacagcggtatcatcgcgtgctccgatgcgtacacggagcattgtggttggatggagggcggtctgctgagcgcccgtgaggcaagccgtctgctgttgcagcgtatcgccgcggcctagagaccactagttgcggccgctgcag

SEQ ID NO: 21 - DNA, 3052 bp, Artificial Sequence
  VioB, Part K274002
gaattcgcggccgcttctagaggtctcatgggtattctggatttcccgcgtatccacttccgtggctgggcccgtgtcaatgcgccgaccgcgaaccgcgatccgcacggccacatcgatatggccagcaataccgtggcgatggcgggtgagccgttcgacctggcacgccatcctacggagttccaccgtcacctgcgctccctgggtccgcgcttcggcttggatggtcgtgctgacccggaaggcccgttcagcctggccgagggctacaacgctgccggtaacaaccacttttcgtgggagagcgcaaccgttagccacgtgcaatgggatggcggtgaggcggatcgtggtgacggtctggtcggtgctcgtttggcactgtggggtcactacaatgattatctgcgtaccaccttcaatcgtgctcgttgggtcgacagcgacccgacgcgccgtgacgctgcacaaatctatgcgggccaattcaccattagcccggctggtgccggtccgggtacgccgtggctgtttacggcagacattgatgatagccatggtgcacgttggacgcgtggcggccacattgcagagcgtggcggccacttcttggatgaagagtttggtctggcacgcctgtttcagttctctgtgccgaaagatcacccacattttctgtttcacccgggtccgtttgattccgaggcctggcgtcgtctgcaattggctctggaggatgacgacgttctgggtctgaccgtgcaatatgcgttgttcaatatgagcaccccgcctcagccgaacagcccggtttttcacgatatggtcggtgttgtcggtctgtggcgtcgtggtgaactggcgagctacccggctggtcgtctgctgcgtccgcgtcaaccgggtctgggtgacctgaccctgcgcgtcaacggtggtcgcgttgcgctgaatttggcgtgtgccattccgttcagcactcgtgccgcgcagccaagcgcaccggaccgcctgaccccggacctgggtgccaaactgccgctgggcgatctgctgctgcgtgatgaggacggcgcactgttggcacgtgtgccgcaggctctgtaccaagactattggacgaatcacggtattgtggacctgccgctgctgcgcgaaccgcgtggtagcttgaccctgagcagcgaactggcggagtggcgtgagcaagactgggtcacccaaagcgacgcgtctaacctgtacctggaggcaccggatcgccgtcacggtcgctttttccctgagagcatcgcgctgcgcagctactttcgcggtgaagcgcgtgcgcgtccggatatcccgcatcgtatcgagggcatgggcctggtcggcgtcgaatctcgtcaggatggcgacgctgcggaatggcgtctgacgggtctgcgtccgggtccggcacgcattgttctggacgatggtgccgaggcgatccctctgcgtgttctgcctgacgattgggcgctggatgacgcgaccgtcgaagaagtggattacgcctttttgtaccgccacgttatggcgtattacgagctggtgtatccattcatgagcgacaaggtgttttccctggctgatcgttgcaaatgtgaaacgtacgcacgtctgatgtggcagatgtgtgatccgcagaaccgcaacaagtcctattacatgccgagcacccgcgaactgtcggcaccgaaagctcgtttgttcttgaagtatctggcccacgtggaaggccaggcacgcctgcaagcacctccgccagcgggtccggcacgcattgaatctaaagcccagttggcggcagagctgcgtaaagccgtcgacctggagctgtctgtgatgctgcaatacctgtacgcggcgtatagcattccgaactatgcacagggccaacaacgtgttcgtgacggtgcgtggaccgccgagcagctgcaactggcgtgcggtagcggtgaccgtcgccgtgatggcggtattcgtgcagcactgctggaaattgctcatgaagaaatgattcattacctggtcgttaacaacctgctgatggccctgggcgagccgttctacgcgggtgtcccgctgatgggcgaagcggcacgtcaggcgtttggcctggacaccgagttcgctctggaaccgtttagcgaaagcacgctggcacgttttgttcgtctggaatggccgcactttatcccagcaccgggcaaatccatcgcggactgctatgccgccattcgtcaggcgtttttggatctgccggacttgtttggtggcgaggcaggtaagcgtggcggtgaacaccacctgttcctgaatgagctgaccaaccgtgcgcatccgggttatcaactggaagttttcgatcgcgactcggcgctgtttggtattgcatttgtgaccgatcagggcgaaggtggcgctctggacagcccgcactacgaacatagccattttcaacgtctgcgtgaaatgagcgcgcgtatcatggctcaaagcgcaccgttcgaaccggcgctgccggcgttgcgtaatccggttctggatgagagcccgggttgccaacgtgtcgcagacggtcgtgcgcgtgcgctgatggcattgtaccaaggcgtttatgagctgatgtttgcgatgatggcgcagcacttcgccgtgaaaccgctgggtagcttgcgtcgcagccgcctgatgaacgcagcaatcgatctgatgaccggtctgttgcgtccgctgagctgcgcgctgatgaacctgccaagcggcatcgccggtcgcacggccggtccgccgctgccgggtccggttgacacccgtagctatgacgactacgcgctgggctgtcgcatgctggcacgccgttgcgagcgtctgctggagcaggcgagcatgctggaaccgggttggctgccggatgcgcagatggagctgctggatttctatcgtcgccaaatgctggacttggcgtgcggcaaactgagccgcgaggccgcctagagaccactagttgcggccgctgcag

SEQ ID NO: 22 - DNA, 1342 bp, Artificial Sequence
  VioC, Part; K274002
gaattcgcggccgcttctagaggtctcatgggtcgtgcgattatcgttggtggcggcctggcgggtggcctgaccgcgatctacctggcgaagcgtggctacgaagtgcacgtcgtggagaagcgtggtgatcctctgcgcgatctgagctcttacgtggacgttgttagcagccgtgcgatcggcgtgagcatgaccgttcgtggtatcaagagcgttttggctgcgggcattccgcgtgcagagctggatgcgtgtggcgaaccgatcgtggcaatggctttctccgtgggtggtcagtatcgcatgcgcgaactgaagccgttggaggatttccgtccgctgagcttgaaccgtgcggcgtttcaaaagctgctgaacaaatacgcgaacctggcaggcgttcgttactactttgagcataagtgcctggatgttgacctggatggtaagagcgtgttgattcagggcaaagatggtcagccgcagcgtctgcaaggtgacatgattatcggtgcggatggcgcccacagcgccgtccgtcaggcgatgcagagcggcctgcgtcgtttcgagttccagcaaacgttcttccgccatggctacaaaaccctggttttgccggacgcgcaagcactgggttaccgtaaagacacgctgtactttttcggcatggattccggtggcctgttcgcgggtcgtgcggctacgatcccagatggtagcgtcagcatcgccgtttgcctgccgtactcgggtagcccttccctgacgaccaccgacgaaccgacgatgcgtgcgttcttcgatcgttacttcggtggcctgccgcgtgacgcgcgtgacgaaatgctgcgtcagtttctggcgaagccgagcaacgacctgattaacgtgcgctctagcacctttcactataagggtaatgtgctgttgctgggtgatgctgcgcatgcgactgcgccgttcctgggtcagggtatgaacatggcgctggaggacgcccgcacgtttgtcgagctgctggaccgccaccagggcgaccaagacaaagcctttccggagttcacggagctgcgcaaagtccaggcagacgcaatgcaagacatggctcgcgccaactatgacgttttgagctgctcgaacccgatctttttcatgcgtgcgcgttacacgcgttacatgcattccaagtttccgggcctgtatccgccggatatggccgagaaactgtactttacgagcgagccgtacgatcgtctgcaacaaatccagcgtaaacagaatgtttggtacaagattggtcgcgtggcctagagaccactagttgcggccgctgcag

SEQ ID NO: 23 - DNA, 1174 bp, Artificial Sequence
  VioD, Part K274002
gaattcgcggccgcttctagaggtctcatgggtattctggtcattggtgctggtccagctggtctggttttcgcatcccaactgaagcaggcacgccctttgtgggccattgacatcgtggagaagaatgacgagcaagaagtgctgggctggggtgtcgtgctgcctggccgtccgggtcagcacccggcgaacccgctgtcctatctggatgcaccggagcgtctgaatccgcaatttctggaggacttcaaactggtgcatcataatgagccgtccttgatgtccacgggcgttttgttgtgcggcgtggagcgtcgcggtctggttcacgcgctgcgcgataagtgccgcagccaaggcattgctattcgtttcgaaagcccgttgctggaacacggtgagctgccgctggcggactatgatctggtggtcctggctaatggtgttaatcacaaaaccgcgcatttcaccgaggctctggtcccgcaggtggactacggccgcaataagtacatttggtatggcactagccagctgttcgatcagatgaatctggtttttcgtacccatggtaaagatatctttatcgcgcatgcctataagtatagcgataccatgagcacgttcattgtcgaatgtagcgaagagacttacgcacgcgcacgcctgggcgaaatgtccgaagaggcgagcgcagaatacgttgctaaggtgttccaggccgagctgggtggtcacggcctggtgagccagccgggtctgggttggcgtaacttcatgacgttgtctcatgaccgttgtcatgatggtaagttggttctgctgggtgacgcgctgcaaagcggtcactttagcatcggccacggcaccacgatggccgtggtggtggcgcagctgctggttaaagcgctgtgtaccgaagatggtgtgcctgccgcgctgaaacgtttcgaagagcgtgccctgccgctggtgcagttgttccgtggccacgcagacaacagccgcgtttggttcgaaaccgtcgaagagcgcatgcacctgtcctcggcggaatttgtgcaaagcttcgacgcacgccgcaaaagcctgccgccgatgccggaagcactggcgcagaatctgcgttatgctttgcaggcctagagaccactagttgcggccgctgcag

SEQ ID NO: 24 - DNA, 628 bp, Artificial Sequence
  VioE, Part K274002
gaattcgcggccgcttctagaggtctcatgggtaaccgtgagccaccactgttgccagcccgttggagcagcgcctatgtctcttattggagcccgatgctgccggatgaccagctgaccagcggctattgctggttcgactatgaacgtgacatctgtcgtattgacggcctgttcaatccgtggagcgagcgtgatactggttatcgcctgtggatgtcggaggttggtaatgcggccagcggccgtacctggaaacaaaaagtcgcctatggtcgtgagcgtaccgccctgggtgaacagctgtgtgagcgtccgctggatgatgagactggcccttttgccgaattgttcctgccacgcgatgtcctgcgccgtctgggtgcccgtcacattggccgtcgcgtggttctgggtcgcgaagcggacggttggcgttaccagcgcccaggtaaaggtccgagcaccctgtacctggatgcggcgagcggcactccactgcgcatggtcaccggcgatgaagcgtcgcgtgcaagcctgcgtgattttccgaatgtgagcgaggcggagatcccggacgcggttttcgcggccaaggcctagagaccactagttgcggccgctgcag

SEQ ID NO: 25 - DNA, 66 bp, Artificial Sequence
  Pr1000, Part Bby_106, J23100 sequence, synthetic, added Trp leader and RBS, converted to Byte format
gcctttgacggctagctcagtcctaggtacagtgctagcaagttcacgtaggaggacagctatggg

SEQ ID NO: 26 - DNA, 66 bp, Artificial Sequence
  Pr562, Part Bby_107, J23106 promoter, synthetic, added Trp leader and RBS, converted to Byte format
gccttttacggctagctcagtcctaggtatagtgctagcaagttcacgtaggaggacagctatggg

SEQ ID NO: 27 - DNA, 66 bp, Artificial Sequence
  Pr248, Part Bby_108, J23105 sequence, synthetic, added Trp leader and RBS, converted to Byte format
gccttttacggctagctcagtcctaggtactatgctagcaagttcacgtaggaggacagctatggg

SEQ ID NO: 28 - DNA, 66 bp, Artificial Sequence
  Pr150, Part Bby_109, J23114 sequence, added Trp leader and RBS, converted to Byte format
gccttttatggctagctcagtcctaggtacaatgctagcaagttcacgtaggaggacagctatggg

SEQ ID NO: 29 - DNA, 66 bp, Artificial Sequence
  Pr64, Part Bby_110, J23109, added Trp leader and RBS, converted to Byte format
gccttttacagctagctcagtcctagggactgtgctagcaagttcacgtaggaggacagctatggg

SEQ ID NO: 30 - DNA, 73 bp, Artificial Sequence
  PrAraC, Part Bby_114, BBa_K206000
tagcaagatagtccataagattagcggatcctacctgacgctttttatcgcaactctctactgtttctccata

SEQ ID NO: 31 - DNA, 81 bp, Artificial Sequence
  PrlambdaC1, Part Bby_113
gccttatcccttgcggtgatagatatttatcccttgcggtgatagatttaacgtaagttcacgtaggaggacagctatggg

SEQ ID NO: 32 - DNA, 85 bp, Artificial Sequence
  PrLacI, Part Bby_111
gcctataaatgtgagcggataacattgacttgtgagcggataacaagatactgagcacaagttcacgtaggaggacagctatggg

SEQ ID NO: 33 - DNA, 84 bp, Artificial Sequence
  PrTetRo, Part BBy_112
gccttccctatcagtgatagagattgactccctatcagtgatagagatactgagcacaagttcacgtaggaggacagctatggg

SEQ ID NO: 34 - DNA, 49 bp, Artificial Sequence
  termTrpAttr, Part BBy_103
gcctaataaagatacccagcccgcctaatgagcgggctttttttttggg

SEQ ID NO: 35 - DNA, 49 bp, Artificial Sequence
  termNegControl, Part BBy_104
gcctaataaagatacccgcgggctctaatgagcgggctttttttttggg

SEQ ID NO: 36 - DNA, 22 bp, Artificial Sequence
  linkStpRbs
gcctaaggaggacagctatggg

SEQ ID NO: 37 - DNA, 22 bp, Artificial Sequence
  linkNegControl
gcctaacctcctctcgaatggg

SEQ ID NO: 38 - DNA, 22 bp, Artificial Sequence
  EndInverterAtoB, Part BBy_105
tgggaacctcctctcgaagcct

SEQ ID NO: 39 - DNA, 22 bp, Artificial Sequence
  EndInverterBtoA, Part BBy_102
gcctaacctcctctcgaatggg

SEQ ID NO: 40 - DNA, 22 bp, Artificial Sequence
  linkOperon, Part BBy_101
gcctaaggaggacagctatggg

SEQ ID NO: 41 - DNA, 49 bp, Artificial Sequence
  Forward (A overhang)
gccgcttctagaggtctcatgggctgatccttcaactcagcaaaagttc

SEQ ID NO: 42 - DNA, 49 bp, Artificial Sequence
  Reverse (B overhang)
gccgcttctagaggtctcagcctctgatccttcaactcagcaaaagttc

SEQ ID NO: 43 - DNA, 743 bp, Artificial Sequence
  pAB, carrying cassette for BBa_I13521
gaattcgcggccgcttctagaggtctcatgggtgggttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataagcctagagaccactagttgcggccgctgcag

SEQ ID NO: 44 - DNA, 743 bp, Artificial Sequence
  pbA - carries cassette for BBa_I13521
gaattcgcggccgcttctagaggtctcagccttgggttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataatgggagagaccactagttgcggccgctgcag

SEQ ID NO: 45 - DNA, 3019 bp, Artificial Sequence
  pAB.rfp.BsaI
gcctagagaccactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagaggaggtctcatgggaattgtgagcggataacaattgacattgtgagcggataacaagatactgagcacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 46 - DNA, 3019 bp, Artificial Sequence
  pBA.rfp.BsaI
tgggagagaccactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagaggaggtctcagcctaattgtgagcggataacaattgacattgtgagcggataacaagatactgagcacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 47 - DNA, 3021 bp, Artificial Sequence
  pAB.rfp.BbsI
gcctaggtcttcactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagaggagaagaccatgggaattgtgagcggataacaattgacattgtgagcggataacaagatactgagcacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 48 - DNA, 3021 bp, Artificial Sequence
  pBA.rfp.BbsI
tgggaggtcttcactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagaggagaagaccagcctaattgtgagcggataacaattgacattgtgagcggataacaagatactgagcacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 49 - DNA, 3025 bp, Artificial Sequence
  pAB.rfp.BfuA1
gccttggtgcaggtactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagaggaacctgcaccatgggaattgtgagcggataacaattgacattgtgagcggataacaagatactgagcacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 50 - DNA, 3025 bp, Artificial Sequence
  pBA.rfp.BfuA1
tgggtggtgcaggtactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacttgacgtctaagaaaccattattatcatgacattaacctataaaaataggcgtatcacgaggcagaatttcagataaaaaaaatccttagctttcgctaaggatgatttctggaattcgcggccgcttctagaggaacctgcaccagcctaattgtgagcggataacaattgacattgtgagcggataacaagatactgagcacatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 51 - DNA, 30 bp, Artificial Sequence
  Forward (BBy.Vf)
gatttctggaattcgcggccgcttctagag

SEQ ID NO: 52 - DNA, 25 bp, Artificial Sequence
  Reverse (BBy.Vr)
cggactgcagcggccgctactagta

SEQ ID NO: 53 - DNA, 20 bp, Artificial Sequence
  sticky end
ttttttttttttttttttaa

(19) United States
(12) Patent Application Publication
(10) Pub. No.: US 2012/0115208 A1
(43) Pub. Date: May 10, 2012

(54) MODULAR METHOD FOR RAPID ASSEMBLY OF DNA

(75) Inventors: Michael ELLISON, Edmonton (CA); Douglas RIDGWAY, Edmonton (CA); Karina ARNESEN, Edmonton (CA)

(73) Assignee: THE GOVERNORS OF THE UNIVERSITY OF ALBERTA, Edmonton (CA)

(21) Appl. No.: 13/282,294

(22) Filed: Oct. 26, 2011

Related U.S. Application Data
(60) Provisional application No. 61/406,673, filed on Oct. 26, 2010.

(51) Int. Cl. C12N 1/21; C12N 15/70; C12N 15/11

(52) U.S. Cl. 435/252.33; 536/23.1; 435/320.1

(57) ABSTRACT

The invention is directed to methods, kits and compositions using specially designed nucleic acid components for efficient assembly of a DNA construct. The method involves a) incubating a support with a first form of nucleic acid components under conditions to form support-bound nucleic acid component complexes; b) removing unbound first form nucleic acid components; c) incubating the support-bound first form nucleic acid component complexes with a second form of nucleic acid components under conditions to anneal and link the second form to the first form; d) removing unbound second form nucleic acid components; e) repeating steps c) and d) until the DNA construct is generated; and f) eluting the DNA construct from the support. The first and second forms of the nucleic acid component comprise sticky ends such that each form cannot link to itself but can link to each other to form an alternating head to tail sequence.

### FIELD OF THE INVENTION

The invention relates to methods, kits and compositions for the efficient assembly of a desired DNA plasmid or construct.

### BACKGROUND OF THE INVENTION

Synthetic biology combines science and engineering to design and construct novel biological entities such as genes, enzymes, and cells, or to redesign existing biological systems. Driven by technical and economic advances in the chemical synthesis of DNA and the assembly of DNA into large constructs, the discipline aims to enable biology as a constructive discipline. The critical technology here is the manipulation of DNA, the genetic code of cells.

Efforts have been made to develop sophisticated techniques to synthesize and assemble increasing lengths of DNA, recently reaching the genome scale with a 538 kb microbial chromosome (Can et al., 2009; Ellis et al., 2011). Such advances are turning genomics from an observational science of studying organisms provided by nature into a hypothesis-driven experimental science, where the DNA content of the genome is as controllable as the bits in a computer. This approach is being used in areas as diverse as labelling with fluorescent proteins to enable visualization, modification of regulatory networks to piece out interactions, gene knockouts for understanding of metabolic networks, and the creation of new disease models, among many others. In addition to the scientific advances enabled, there are obvious applications in areas such as health, biofuels, agriculture, chemical production, the environment, and biosensors.

Regardless of the ease of construction, limiting factors for engineering purposes include existing biological knowledge and the ability to predict the behavior of the newly designed systems. A proposed solution to such challenges is modularity, where individual genetic components are defined and characterized, and linked together in standardized, well defined ways, hiding the underlying complexity behind a defined interface (Endy, 2005; Arkin, 2008). The BioBricks Foundation is an organization founded by engineers and scientists from MIT, Harvard, and UCSF who develop and encourage the use of technologies based on BioBrick™ standard biological parts that encode basic biological functions. Examples of BioBrick™ parts include promoters, ribosome-binding sites, coding sequences and transcriptional terminators. The Registry of Standard Biological Parts contains more than 3000 parts of varying degrees of characterization, all of which can be combined through standard molecular biology techniques.

A method of in vitro DNA construction is based on conventional directional cloning and standardizes the restriction sites and order of procedures, allowing a single BioBrick™ to be added at either the 5′ (head) or 3′ (tail) of another BioBrick™. Although this method is useful, it is labor intensive and time-consuming, requiring the plasmids containing each BioBrick™ part to be amplified by transformation into bacteria, growth of an overnight bacterial culture, and plasmid purification. Standard assembly also requires the performance of tedious restriction enzyme digestions, gel electrophoresis, purification of the digested DNA fragments, and ligation reactions. Each of these methods leaves a scar sequence that is not always benign. A major disadvantage of the BioBrick™ method is the restriction on the DNA sequence to be assembled. Since the standardized ends are based on a number of relatively common 6-cutter restriction enzymes, the BioBrick™ assembly cannot process DNA sequences containing any of these sequences as internal restriction sites. In the process of “BioBricking,” an existing sequence typically requires removal of one or more restriction sites, necessitating rounds of site-directed mutagenesis or even from-scratch chemical gene synthesis to a sequence designed with BioBrick™ constraints in mind (Shetty et al., 2008).

Alternative or related methods to BioBrick™ have been proposed (Ellis et al., 2011) including, for example, BglBricks (Anderson et al., 2010), In-Fusion™ cloning (Sleight, 2010), and BioBricks Foundation RFCs. These approaches adjust the restriction sites and the resulting scars formed, easing the construction of protein fusions, but do not address assembly speed or limitations on DNA sequences. Alternative approaches of cloning include, for example, Gateway™ (Hartley et al., 2000), sequence and ligation independent cloning (SLIC) (Li and Elledge, 2007), USER™ (Bitinaite et al., 2007), and SOE™ (Heckman et al., 2007). However, such approaches are not modular. Further, the USER™ enzymes are capable of inducing damage, resulting in non-ligatable DNA.

Accordingly, there is thus a need in the art for the development of improved efficient and reliable systems for assembly of nucleic acid constructs.

### SUMMARY OF THE INVENTION

The present invention relates to methods, kits and compositions for the efficient assembly of a DNA construct, such as a plasmid.

In one aspect, the invention provides a method for assembly of a DNA construct comprising the steps of:

a) incubating a support with a first form of nucleic acid components under conditions to form support-bound nucleic acid component complexes;

b) removing unbound first form nucleic acid components;

c) incubating the support-bound first form nucleic acid component complexes with a second form of nucleic acid components under conditions to anneal and link the second form to the first form;

d) removing unbound second form nucleic acid components;

e) repeating steps c) and d) until the DNA construct is generated; and

f) eluting the DNA construct from the support;

wherein the first and second forms of nucleic acid component comprises sticky ends such that each form cannot link to itself but can link to each other to form an alternating head to tail sequence.

In one embodiment, the sticky end is nonpalindromic. In one embodiment, the sticky ends comprise sequences within a predetermined set of sequences. In one embodiment, the sticky end comprises a sequence as set forth in any one of SEQ ID NOS: 53 to 71. In one embodiment, the complementary forms comprise SEQ ID NOS: 53 and 54. In one embodiment, the complementary forms comprise SEQ ID NOS: 55 and 56. In one embodiment, the nucleic acid component comprises SEQ ID NO: 53 at one end, and SEQ ID NO: 56 at the other end. In one embodiment, the nucleic acid component comprises SEQ ID NO: 55 at one end, and SEQ ID NO: 54 at the other end. In one embodiment, the sticky end has a length of about 4 base pairs.

In one embodiment, the nucleic acid component further comprises one or more nucleic acid sequences providing one or more biological functionalities. In one embodiment, the nucleic acid component encodes a biological functionality comprising one or more of origin of replication, selectable marker, transcriptional regulatory element, structural gene or fragment thereof, transcription termination signal, translational regulatory sequence, regulators of mRNA stability, cellular localization signal, recombination elements, mutagenized genes, protein domain encoded regions, synthetic multiple cloning sites, unique restriction enzyme or DNA cleavage sites, and site for covalent or non covalent attachment of a biological or chemical molecule.

In one embodiment, the nucleic acid sequence provides an open reading frame lacking initiation and termination codons. In one embodiment, the nucleic acid sequence provides a ribosome binding site, initiation and termination codons, and a linker for an open reading frame.

In one embodiment, the nucleic acid component comprises a sequence as set forth in any one of SEQ ID NOS: 1 to 40.

In one embodiment, the nucleic acid component comprises an anchor sequence annealed or covalently bound to the support.

In one embodiment, the anchor sequence comprises a 5′ sticky poly-dA, a Type IIs restriction site, and a 3′ terminal sequence. In one embodiment, the 3′ terminal sequence comprises a sequence selected from 5′-TGGG or 5′-GCCT. In one embodiment, the support comprises a bead or microsphere capable of binding the anchor sequence. In one embodiment, the nucleic acid component comprises a terminator sequence comprising a poly-dT end cap. In one embodiment, the nucleic acid component comprises a direction reversing linker.

In one embodiment, the nucleic acid components are incubated in a step-wise manner. In one embodiment, the nucleic acid components are incubated simultaneously. In one embodiment, the elution of step (f) comprises treatment with heat, an elution buffer, or both. In one embodiment, the elution buffer comprises a sodium hydroxide solution. In one embodiment, the method further comprises transforming a host cell with the eluted DNA construct. In one embodiment, the host cell comprises an E. coli cell. In one embodiment, the DNA construct comprises a size greater than 1 kb.

In another aspect, the invention provides a kit for assembly of a DNA construct comprising a first form and a second form of nucleic acid components, each component comprising double-stranded DNA having sticky ends to allow for annealing and linking of the nucleic acid components in a predetermined order to generate the DNA construct, wherein the first and second forms of nucleic acid component comprises sticky ends such that each form cannot link to itself but can link to each other to form an alternating head to tail sequence. In one embodiment, the kit comprises a sequence as set forth in any one of SEQ ID NOS: 1-40 and 45-50.

In another aspect, the invention provides a composition comprising one or more nucleic acid components as set forth in any one of SEQ ID NOS: 1-40 and 45-50.

In another aspect, the invention provides a vector comprising a sequence as set forth in any one of SEQ ID NOS: 45-50.

In yet another aspect, the invention provides a method of preparing the above nucleic acid component comprising the steps of:

a) selecting a double-stranded nucleic acid molecule; and

b) generating sticky ends to the double-stranded nucleic acid molecule to produce the nucleic acid component.

In one embodiment, step (b) comprises the step of:

a) introducing a double stranded nucleic acid into a vector wherein digestion with a restriction enzyme releases the nucleic acid component with the desired sticky ends; or

b) conducting PCR-amplification of a linear fragment comprising restriction sites using a plasmid comprising the same restriction sites wherein digestion with one or more restriction enzymes releases the nucleic acid component with the desired sticky ends; or

c) generating a plurality of DNA oligos and annealing the oligos to produce the nucleic acid component having sticky ends; or

d) generating a heteroduplex from a pair of polynucleotides, the heteroduplex comprising sticky ends to produce the nucleic acid component.

In one embodiment, the vector comprises a sequence as set forth in any one of SEQ ID NOS: 45-50. In one embodiment, the method further comprises purifying the nucleic acid component by gel electrophoresis, HPLC, or solid phase adsorption. In one embodiment, purification is conducted in a binding buffer comprising GuHCl, KCl, Tris-HCl and MgCl₂.

In yet another aspect, the invention comprises a nucleic acid component formed by the above method.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings:

FIG. 1 is a schematic diagram of one embodiment of the method of the present invention.

FIG. 2 is a schematic diagram of one embodiment of the method of the present invention.

FIG. 3 is a photograph of an electrophoretic gel demonstrating the fidelity and efficiency of AB to BA component ligation.

FIG. 4 is a schematic diagram illustrating how the A and B regions affect N- and C-terminal codons for open reading frame parts used in isolation (upper) and as part of protein fusions (lower).

FIG. 5 is a schematic diagram of a strategy to prepare a byte using a plasmid flanked by suitable Type IIs restriction sites.

FIG. 6 is a schematic diagram of spontaneous circularization using poly-dT end cap to form a plasmid.

FIG. 7 is a schematic diagram of one embodiment of the method of the present invention.

FIG. 8A is a schematic diagram of a strategy to construct an octomer.

FIG. 8B is a photograph of an electrophoretic gel with lanes as 1 kb+ ladder (Invitrogen), tetramer (x4) and octomer (x8).

FIG. 9 is a representative chromatogram showing separation of DNA molecule from cleaved flanking sequences.

FIG. 10 shows the sequence of the pAB.rfp.BsaI plasmid (SEQ ID NO: 45).

FIG. 11 shows the sequence of the pBA.rfp.BsaI plasmid (SEQ ID NO: 46).

FIG. 12 shows the sequence of the pAB.rfp.BbsI plasmid (SEQ ID NO: 47).

FIG. 13 shows the sequence of the pBA.rfp.BbsI plasmid (SEQ ID NO: 48).

FIG. 14 shows the sequence of the pAB.rfp.BfuA1 plasmid (SEQ ID NO: 49).

FIG. 15 shows the sequence of the pBA.rfp.BfuA1 plasmid (SEQ ID NO: 50).

FIG. 16 is a schematic diagram of constructs assembled and transformed into DH5α E. coli cells, and results of the transformation as indicated on an electrophoretic gel.

FIG. 17 are photographs of electrophoretic gels comparing the coupling efficiency of an annealed anchor (left gel) and a covalently bound anchor (right gel).

FIG. 18 shows gene synthesis errors in a synthetic fragment. The division into synthetic oligos is indicated by case, and the locations of mutations across all sequenced constructs are indicated by asterixes.

FIG. 19A is a schematic diagram of a strategy to prepare a heteroduplex from two linear PCR products.

FIG. 19B is a photograph of an electrophoretic gel showing the results of dimerization by ligating an excess of 0.7 kb AB part with 1.2 kb Anchor-A′ part. Lane 1: AB part produced by enzymatic digestion and HPLC separation. Lane 2: AB part produced by heteroduplexing linear PCR products. In both cases, the Anchor-A′ part is completely consumed, demonstrating the presence of a functional A end. Lane 3: Invitrogen 1 kb Plus ladder.

FIG. 20 is a photograph of an electrophoretic gel showing the results of buffer optimization for spin column purification.

### DETAILED DESCRIPTION OF PREFERRED EMBODIMENTS

The present invention relates to methods, kits and compositions for the efficient assembly of a DNA construct.

When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims. To facilitate understanding of the invention, the following definitions are provided:

An “alternating head to tail sequence” refers to the alternating arrangement of bytes which are constructed in two forms; for example, an “AB” form and a “BA” form. Each form has incompatible ends, meaning that neither form can be linked to itself. However, the ends of each form are compatible with the other form, allowing for alternating order of AB and BA forms in a head-to-tail orientation. Adding bytes to the growing chain by alternating the AB and BA forms ensures that only one copy of each is added at each step.

A “biological functionality” is meant to include, but is not limited to, an origin of replication, selectable marker, transcriptional regulatory element, structural gene or fragment thereof, transcription termination signal, translational regulatory sequence, regulators of mRNA stability, cellular localization signal, recombination elements, mutagenized genes, protein domain encoded regions, synthetic multiple cloning sites, unique restriction enzyme or DNA cleavage sites, and site for covalent or non covalent attachment of a biological or chemical molecule.

A “coding sequence” or “coding region” or “open reading frame (ORF)” is part of a gene that codes for an amino acid sequence of a polypeptide.

A “complementary sequence” is a sequence of nucleotides which forms a duplex with another sequence of nucleotides according to Watson-Crick base pairing rules where “A” pairs with “T” and “C” pairs with “G.”

A “construct” is a polynucleotide which is formed by polynucleotide segments isolated from a naturally occurring gene or which is chemically synthesized. The “construct” is combined in a manner that otherwise would not exist in nature, and is usually made to achieve certain purposes. For instance, the coding region from “gene A” can be combined with an inducible promoter from “gene B” so the expression of the recombinant construct can be induced. The term should be understood to include a plasmid.

“Nonpalindromic” means a sequence in double-stranded nucleic acids that does not read the same on both strands when reading one strand from left to right and the other from right to left (i.e., both strands are read 5′ to 3′).

“Nucleic acid” means polynucleotides such as deoxyribonucleic acid (DNA), and, where appropriate, ribonucleic acid (RNA). The term should also be understood to include, as equivalents, analogs of either RNA or DNA.

“Plasmid” means a DNA molecule which is separate from, and can replicate independently of, the chromosomal DNA. They are double stranded and, in many cases, circular. Plasmids used in genetic engineering are known as vectors and are used to multiply or express particular genes.

A “polynucleotide” is a linear sequence of ribonucleotides (RNA) or deoxyribonucleotides (DNA) in which the 3′ carbon of the pentose sugar of one nucleotide is linked to the 5′ carbon of the pentose sugar of another nucleotide. The deoxyribonucleotide bases are abbreviated as “A” deoxyadenine; “C” deoxycytidine; “G” deoxyguanine; “T” deoxythymidine; “I” deoxyinosine. Some oligonucleotides described herein are produced synthetically and contain different deoxyribonucleotides occupying the same position in the sequence. The blends of deoxyribonucleotides are abbreviated as “W” A or T; “Y” C or T; “H” A, C or T; “K” G or T; “D” A, G or T; “B” C, G or T; “N” A, C, G or T.

A “polypeptide” is a sequence of amino acids linked by peptide bonds. The amino acids are abbreviated as “A” alanine; “R” arginine; “N” asparagine; “D” aspartic acid; “C” cysteine; “Q” glutamine; “E” glutamic acid; “G” glycine; “H” histidine; “I” isoleucine; “L” leucine; “K” lysine; “M” methionine; “F” phenylalanine; “P” proline; “S” serine; “T” threonine; “W” tryptophan; “Y” tyrosine and “V” valine.

A “vector” is a polynucleotide that is able to replicate autonomously in a host cell and is able to accept other polynucleotides. For autonomous replication, the vector contains an “origin of replication.” The vector usually contains a “selectable marker” that confers the host cell resistance to certain environment and growth conditions. For instance, a vector that is used to transform bacteria usually contains a certain antibiotic “selectable marker” which confers the transformed bacteria resistance to such antibiotic.

The present invention relates to an efficient and rapid method of producing multi-component DNA plasmids or constructs using specially designed nucleic acid components. As used herein, the term “nucleic acid component” means a basic unit of assembly used in the present invention. These units are comprised of nucleic acid molecules, preferably double-stranded DNA, which have standardized sticky ends for assembling the nucleic acid components into a desired DNA construct. The nucleic acid sequences contained within each nucleic acid component provide the requisite information for a specific biological function(s) or for a specific utility. Examples of nucleic acid components include nucleic acid sequences which encode a polypeptide, include an origin of replication, and/or include a selectable marker, alone or in combination with other biologically active nucleotide sequences.

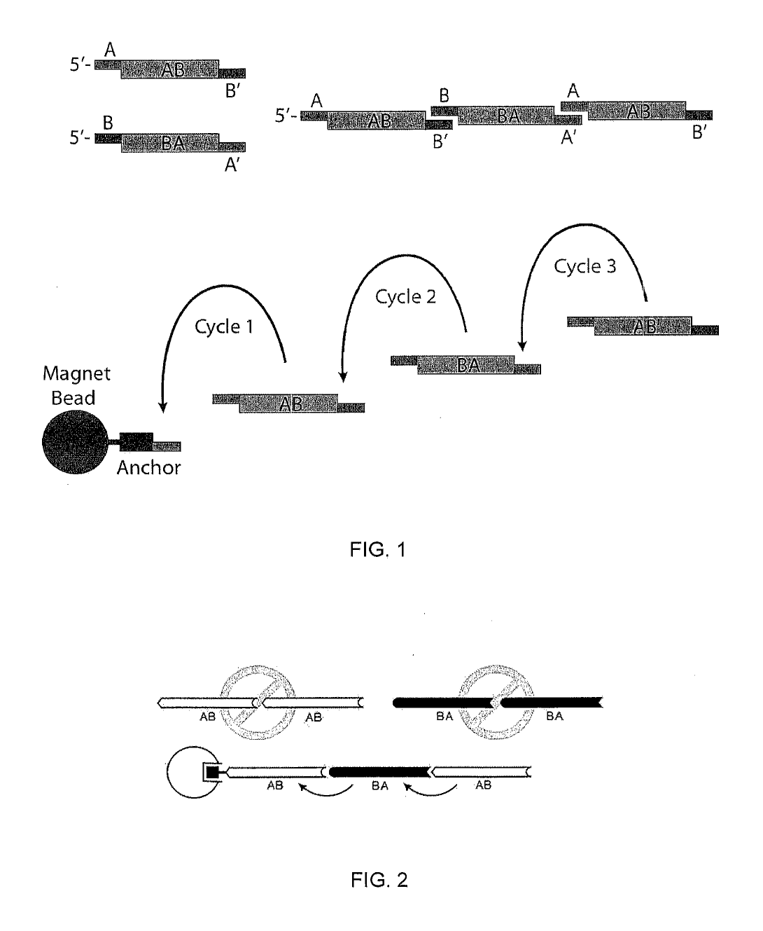

The assembly is accomplished using a support, preferably a solid support, including, but not limited to, a bead or microsphere as are well known in the art. In one embodiment, the support comprises an oligo-dT paramagnetic bead. Paramagnetic beads facilitate pelleting and solution changes during the washing steps as described in Example 4. Using a bead- or microsphere-linked assembly of prepared nucleic acid components is efficient and convenient, since the desired DNA plasmid or construct can be assembled in hours rather than days, compared to conventional DNA construction methods which require lengthy intermediate cloning steps and transformations. The method is shown generally in FIG. 1 comprising the steps of incubation or binding, washing and elution.

In one embodiment, the invention provides a method for assembly of a DNA construct comprising the steps of:

a) incubating a support with a first form of nucleic acid components under conditions to form support-bound nucleic acid component complexes;

b) removing unbound first form nucleic acid components;

c) incubating the support-bound first form nucleic acid component complexes with a second form of nucleic acid components under conditions to anneal and link the second form to the first form;

d) removing unbound second form nucleic acid components;

e) repeating steps c) and d) until the DNA construct is generated; and f) eluting the DNA construct from the support;

wherein the first and second form of nucleic acid component comprises sticky ends such that each form cannot link to itself but can link to each other to form an alternating head to tail sequence.

In one embodiment, the method comprises sequential assembly of the nucleic acid components on the support to generate the DNA construct. It will be recognized by those skilled in the art that typical problems in sequential assembly include ensuring directionality of each added fragment and controlling the copy number of the added fragment. Use of nonpalindromic sticky ends ensures directionality since each added fragment can join in only one orientation; however, use of the same ends on each fragment leaves no control over the copy number.

Accordingly, the assembly of the DNA construct is achieved by employing nucleic acid components comprising nucleic acid molecules, preferably double-stranded DNA, which include specific terminal sequences (referred to as “sticky ends”) required for assembling the nucleic acid components into a DNA construct. The nucleic acid components are designated herein as “bytes.” As shown in FIG. 2, each byte is constructed in two forms: an “AB” form and a “BA” form. Each form has incompatible ends, meaning that neither form can be linked to itself. However, the ends of each form are compatible with the other form, allowing for alternating order of AB and BA forms in a head-to-tail orientation. Adding bytes to the growing chain by alternating the AB and BA forms ensures that only one copy of each is added at each step (FIG. 2). The invention thus enables modularity of assembly such that a single unit, such as an AB fragment, can be placed into a variety of constructs in a variety of locations, provided that the AB-BA alteration is respected. Constructs can thus be assembled from modular, reusable parts. In one embodiment, a single unit comprises approximately 1 kbp. In one embodiment, an assembled construct comprises greater than twenty thousand base pairs.

Upon completion of the desired product, the chain can be released from the support by a chemical cleavage to yield the desired linear construct. Alternatively, an annealed terminator may be added to the chain to allow circularization upon release of the construct from the support. The circularized constructs can then be introduced into living cells and propagated, provided an origin of replication was included during construction.

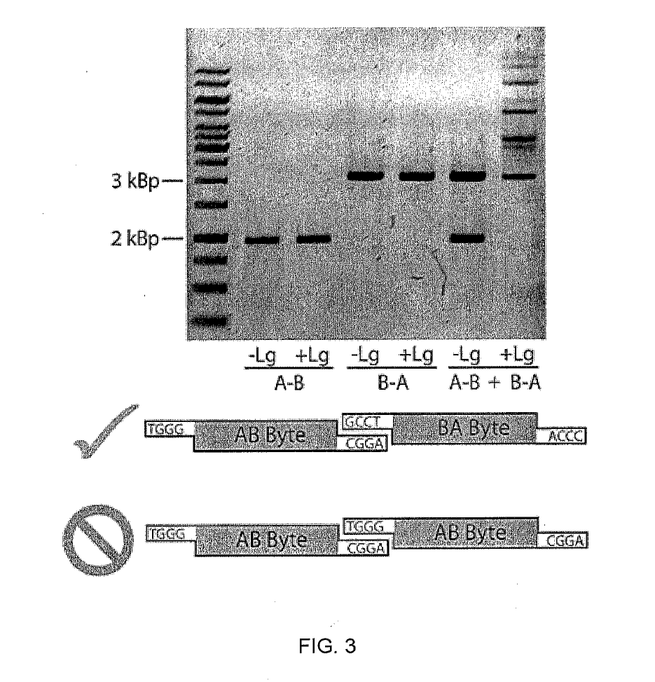

In one embodiment, the terminal sequences or sticky ends are nonpalindromic. The sticky ends are designated in FIG. 1 as “A” and “B.” The “AB” and “BA” bytes comprise double-stranded DNA flanked by 5′ sticky ends of any suitable length. In one embodiment, the 5′ sticky ends are about 4 bp in length. The 5′ sticky ends are designed so that there is little cross annealing between A and B sequences, but good annealing between sequence A and its reverse complement A′, and likewise between B and its reverse complement B′. Thus, the AB byte has two 5′ sticky ends, one having sequence A and the other having sequence B′. The BA byte has two 5′ sticky ends, one having sequence B and the other having sequence A′. The A and A′ ends anneal and ligate. Similarly, the B and B′ ends anneal and ligate. A does not anneal with A, B or B′, and similarly for all ends. The fidelity and efficiency of annealing with these sequences were confirmed, as shown in FIG. 3.

In one embodiment, the 5′ sticky ends (A, A′, B, B′) have the sequences set forth in Table 1.

TABLE 1
Sequences of the 5′ sticky ends

| | | |
|---|---|---|
| A | 5′-TGGG | SEQ ID NO: 53 |
| A′ | 5′-CCCA | SEQ ID NO: 54 |
| B | 5′-GCCT | SEQ ID NO: 55 |
| B′ | 5′-AGGC | SEQ ID NO: 56 |

In one embodiment, the sticky ends have the sequences set forth in Table 2. The sticky ends have either a 5′ or 3′ overhang as indicated. Only the overhang is shown. Duplex DNA beyond the overhang is indicated by ellipses ( . . . ). The appropriate cognates for each end, consisting of the reverse complement of the sequence for each end with the same 5′ or 3′ nature, were also tested but are

TABLE 2
Sequences of the sticky ends

| | |
|---|---|
| 5′-TGGG . . . | SEQ ID NO: 57 |
| 5′-GCCT . . . | SEQ ID NO: 58 |
| 5′-CGTT . . . | SEQ ID NO: 59 |
| 5′-GAAG . . . | SEQ ID NO: 60 |
| 5′-GCGA . . . | SEQ ID NO: 61 |
| 5′-ATGG . . . | SEQ ID NO: 62 |
| 5′-CTGA . . . | SEQ ID NO: 63 |
| . . . TGCT-3′ | SEQ ID NO: 64 |
| . . . ACAA-3′ | SEQ ID NO: 65 |
| . . . ATCC-3′ | SEQ ID NO: 66 |
| . . . AACA-3′ | SEQ ID NO: 67 |
| . . . CATC-3′ | SEQ ID NO: 68 |
| . . . GCCT-3′ | SEQ ID NO: 69 |
| . . . ATGC-3′ | SEQ ID NO: 70 |
| . . . TTTTTTTTTTTTTTTTTTAA-3′ | SEQ ID NO: 71 |

As will be understood by those skilled in the art, the terms 5′ and 3′ are used to describe the ends of the duplex DNA. One strand may be identified arbitrarily as the “top” strand; thus, the two ends are identified based on whether they are the 5′ or 3′ end of the “top” strand. The term is also meant to refer to the type of overhang for which there are two possibilities: a 5′ overhang (which is the same as a 3′ recessed) and a 3′ overhang (which is the same as 5′ recessed). A 5′ or a 3′ overhang may be present at either the 5′ or the 3′ ends of a duplex DNA. A sticky end sequence alone is not sufficient to determine complementarity since the overhang must also be considered.

In one embodiment, restriction enzymes are used to generate the standardized sticky ends. In one embodiment, the restriction enzyme comprises a Type IIs restriction enzyme. The 5′ sticky ends are produced by digestion with a Type IIs restriction enzyme oriented to cut leaving the sticky end, but eliminating the restriction site recognition sequence. Suitable enzymes include, but are not limited to, BsaI, BbsI, BfuAI, BbvI, BsmAI, BspMI, FokI, SfaNI, AarI, BtgZI, Esp3I, FaqI and isoschizomers.

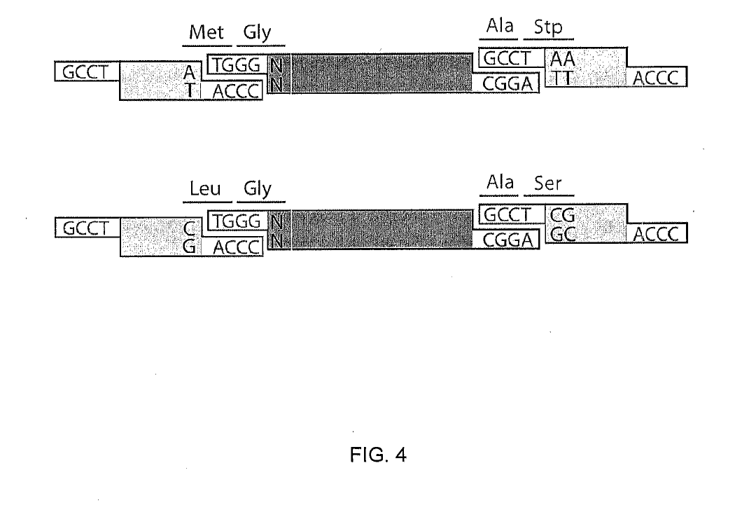

The nucleic acid sequences contained within each nucleic acid component provide the requisite information for a specific biological function(s) or for a specific utility, and impact the resultant DNA plasmid or construct and the protein encoded thereby. As shown in FIG. 4, AB region choice affects protein termini and fusions. In one embodiment, AB bytes provide the open reading frames (ORFs), without initial methionine or final stop codons, allowing the assembly of protein fusions. A BA linker intended to initiate an amino acid will end with an alanine, giving Met-Gly as the first two amino acids of the chain. The B region codes for alanine, allowing the chain to be terminated with an alanine-Stop if the next linker is intended to terminate, of Ala-Ser if the next linker is being used to continue the fusion. In one embodiment, BA bytes provide the ribosome binding site, the initial start codon, the terminator, and a linker to the next ORF for making a protein fusion. Other functions usefully encoded in BA bytes include, but are not limited to, promoters, operators, N- and C-terminal tags, peptide linkers, gene spacers, RNA terminators and linkers. The present invention thus provides a choice of building fusions, operons, or individually regulated protein units, simply by adjusting which specific parts are used to link the ORFs. The specific choice of A and B regions impacts the N- and C-terminal amino acids, so these regions have been designed to give acceptable options.

The nucleic acid component may thus encode a biological functionality which may include, but is not limited to, an origin of replication, selectable marker, transcriptional regulatory element, structural gene or fragment thereof, transcription termination signal, translational regulatory sequence, regulators of mRNA stability, cellular localization signal, recombination elements, mutagenized genes, protein domain encoded regions, synthetic multiple cloning sites, unique restriction enzyme or DNA cleavage sites, and site for covalent or non covalent attachment of a biological or chemical molecule.

Anchors are bound to the support and are used in turn to bind the first nucleic acid component to initiate the subsequent chain of multiple nucleic acid components forming the DNA construct. The anchor will comprise one sticky end to initiate the chain. For example, in FIG. 1, the anchor comprises an A′ sticky end.

In one embodiment, the anchor is annealed to the support. The anchor comprises a 5′ sticky poly-dA which directly attaches to poly-dT paramagnetic beads without requiring additional chemical steps. The anchor-support structure is robust, but still easily released by heating. Type IIs restriction sites are built into the anchor to allow release of a functional A or B sticky end, enabling hierarchical assembly and recircularization by ligation of A or B ends. In one embodiment, the nucleic acid component comprises an anchor sequence. In one embodiment, the anchor sequence comprises a 5′ sticky poly-dA, a Type IIs restriction site, and a 3′ terminal sequence. In one embodiment, the 3′ terminal sequence comprises a sequence selected from 5′-TGGG or 5′-GCCT. Terminators allow the assembled DNA to circularize into a plasmid after release from the support. Standardized priming sequences are built into both the anchors and terminators to allow ease of sequencing the assembled product. The anchors and terminators may be provided in both A and B end variants for flexibility and enablement of hierarchical assembly as described herein.

In another embodiment, the anchor is covalently bound to the support. As described in Example 7, AB and BA fragments were taken through twenty-one cycles of assembly. Yields were computed from band densitometry and reported on a molar basis, corrected for bead loss. The average coupling efficiency over 21 steps was higher for the covalently bound anchor compared to the annealed anchor (FIG. 17). While the covalent attachment provided an improvement in yield over annealing alone, at very long lengths shearing and/or other effects increase the rate of product loss and limit the total length of construct which can be assembled.

In one embodiment, the invention provides a method of preparing the nucleic acid component comprising the steps of:

a) selecting a double-stranded nucleic acid molecule; and

b) generating sticky ends to the double-stranded nucleic acid molecule to produce the nucleic acid component.

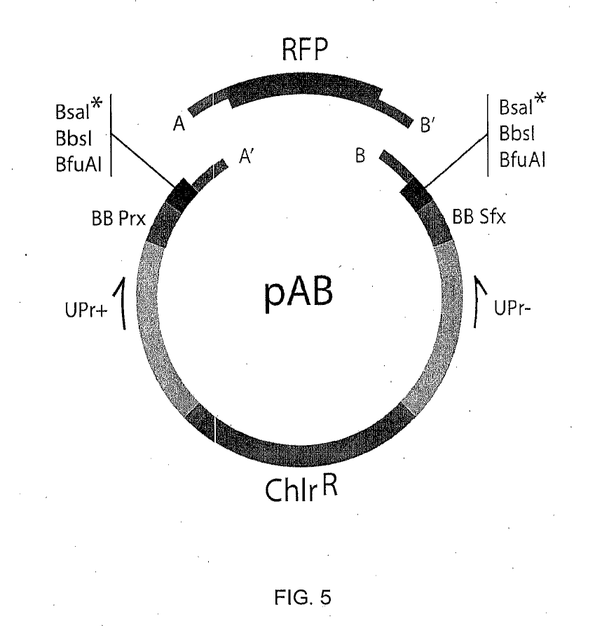

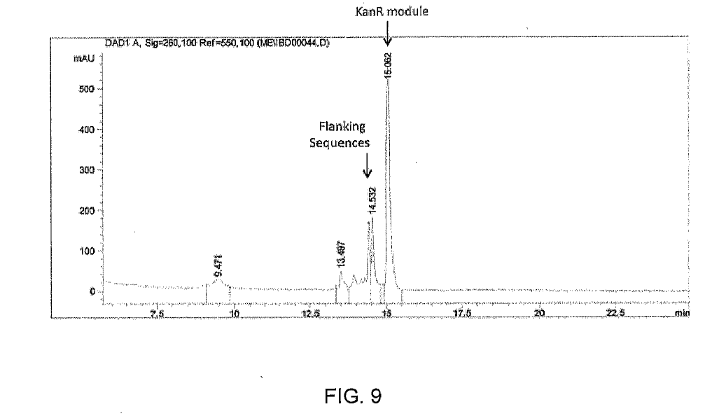

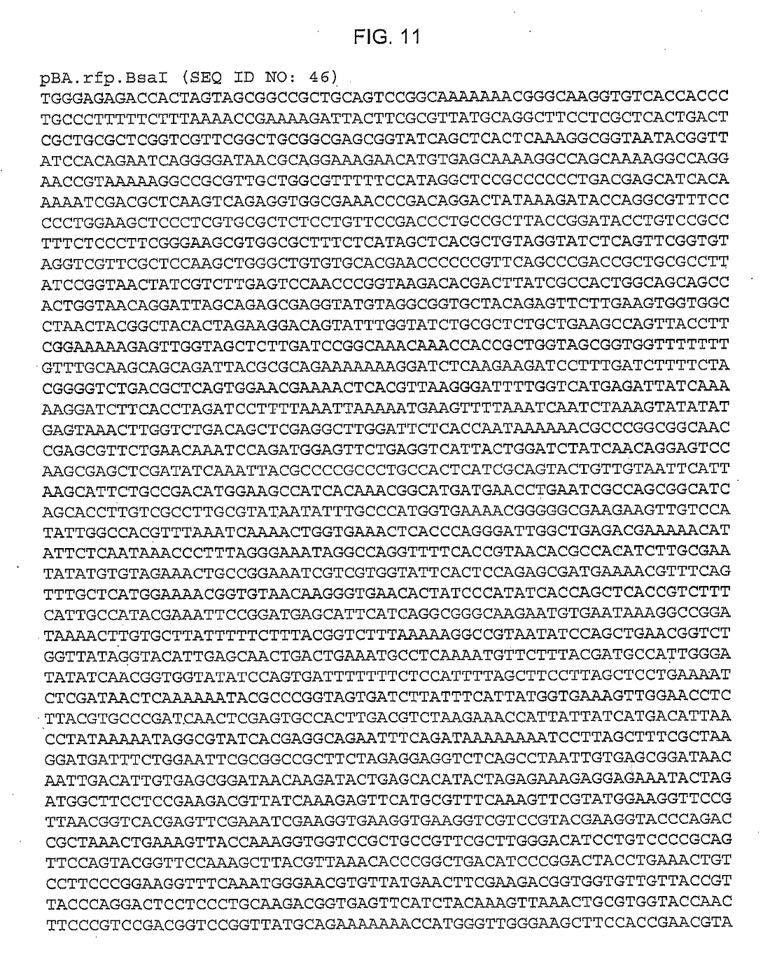

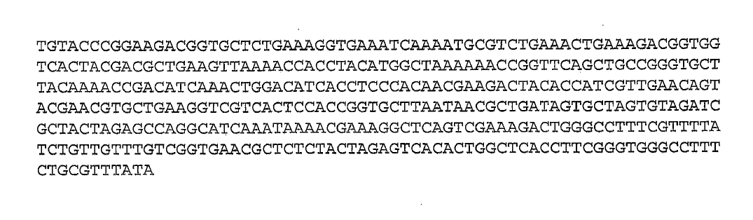

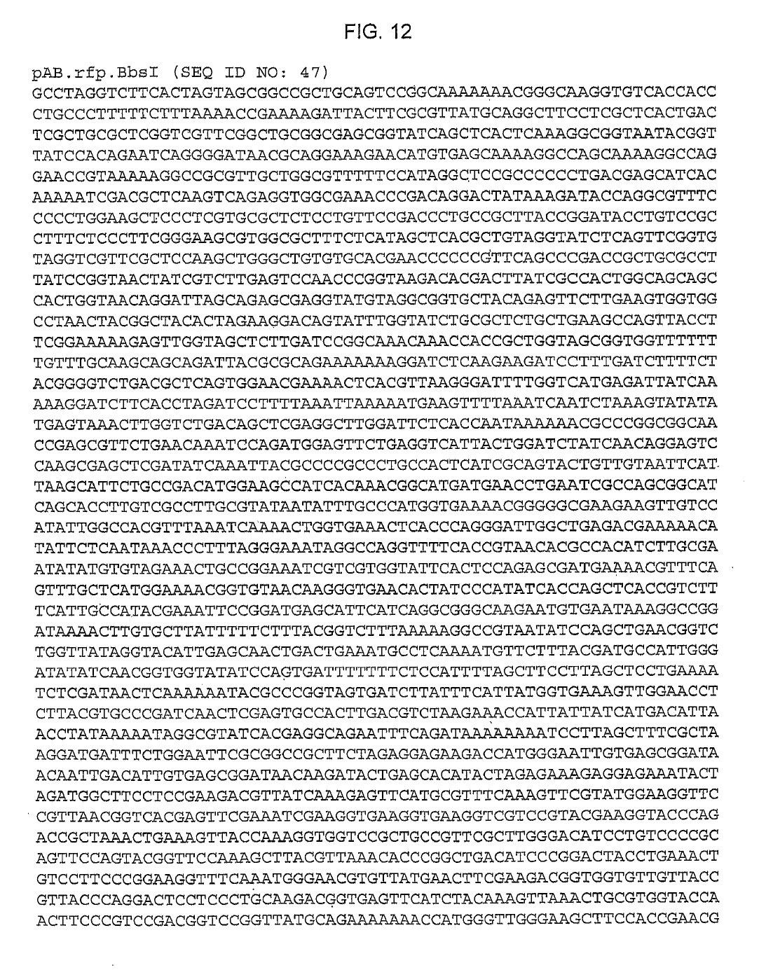

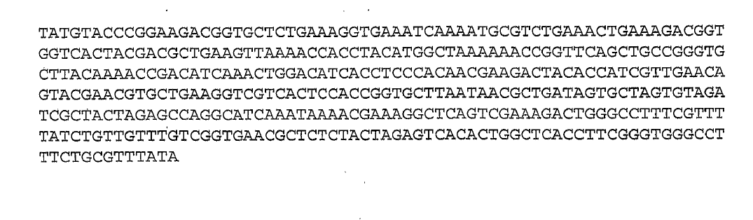

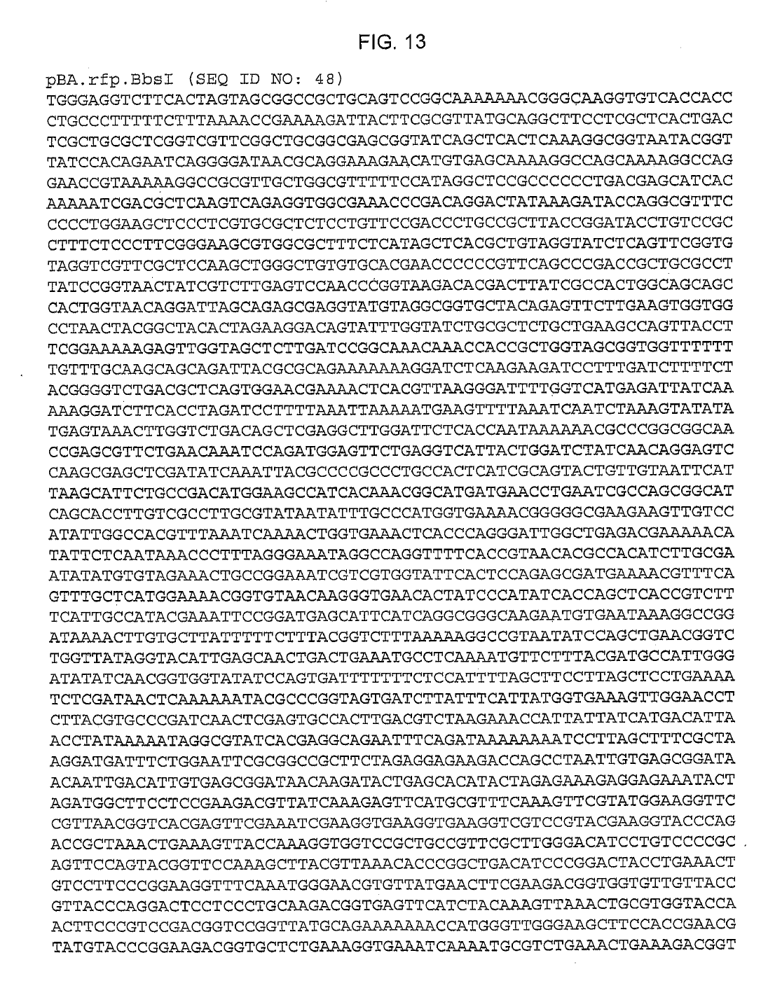

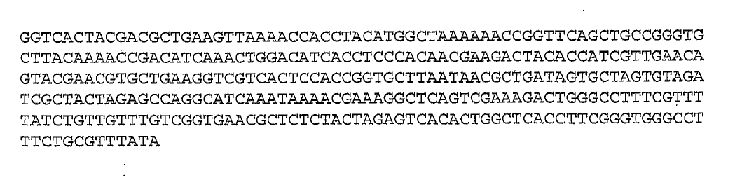

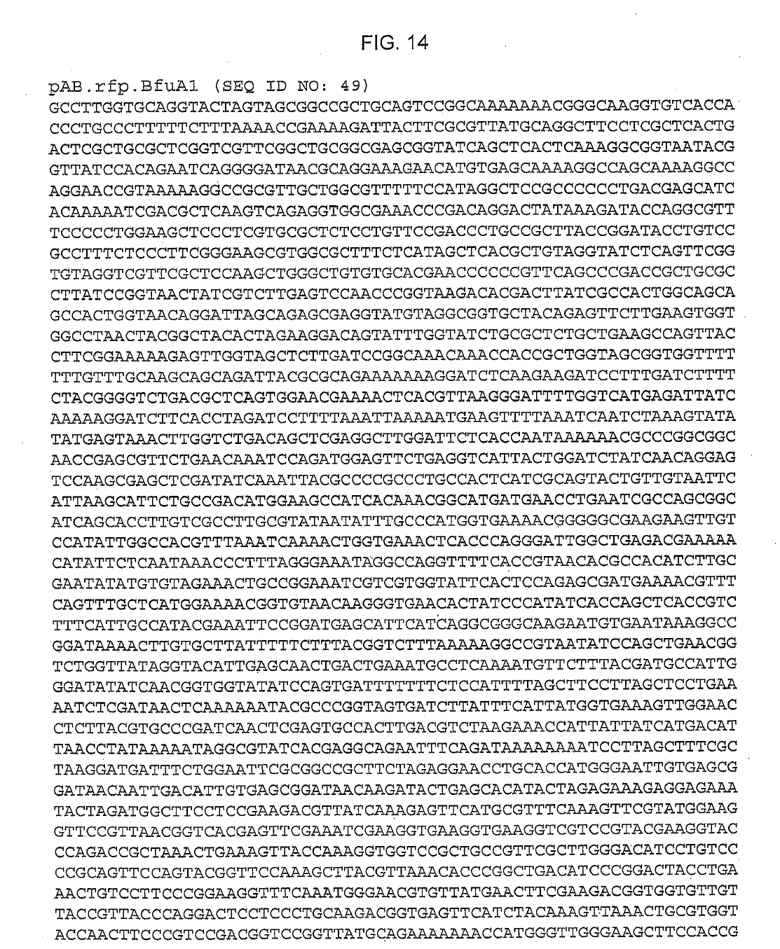

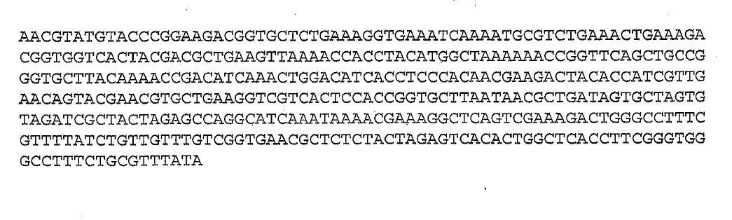

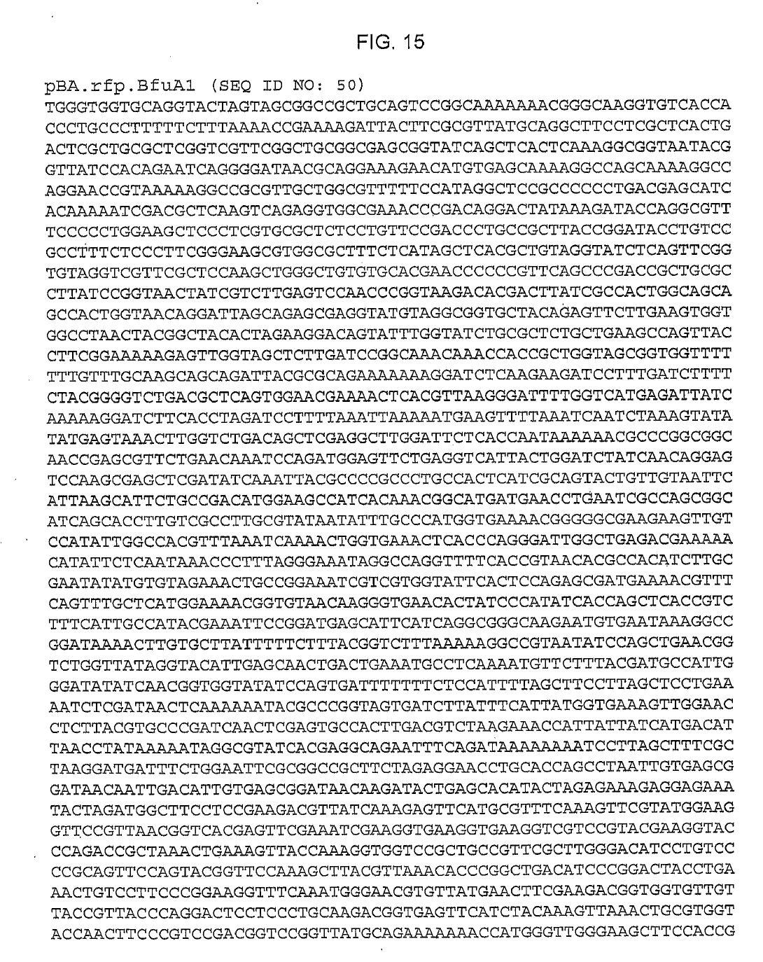

Various methods may be used to prepare the bytes, anchors and terminators for use in the method of the present invention (Examples 1-3 and 6-9, FIGS. 9-18 and 19A-B). In one embodiment, a gene of interest is cloned into a plasmid pAB designed to release the byte with the designed sticky ends after digestion with a Type IIs restriction enzyme. The byte is then purified by gel electrophoresis or HPLC. The Type IIs site chosen must be compatible with the byte sequence, in that the recognition sequence for the enzyme may not appear in the byte sequence. If it does, an alternative Type IIs sequence must be chosen. Alternatively, a linear fragment containing the byte and Type IIs restriction sites is amplified by PCR from a template incorporating the restriction sites, such as the plasmid shown in FIG. 5. FIG. 5 shows release of red fluorescent protein (RFP) in the AB format. Versions of this plasmid with three separate Type IIs restriction sites (BsaI, BbsI, and BfsAI) have been constructed, enabling compatibility with byte contents containing any one or two of those restriction sites. Universal priming sites Upr+ and Upr− may be used for length confirmation, sequencing or production by PCR. After digestion with the restriction enzyme, a simpler method of purification such as a PCR clean-up kit suffices to remove unwanted DNA fragments. For parts which are difficult to clone into a carrier vector, such as plasmid origins, PCR from any template with custom primers including 5′ extensions with the desired A/B sequences and Hs restriction sites, allowing the byte to be released by digestion and PCR cleanup.

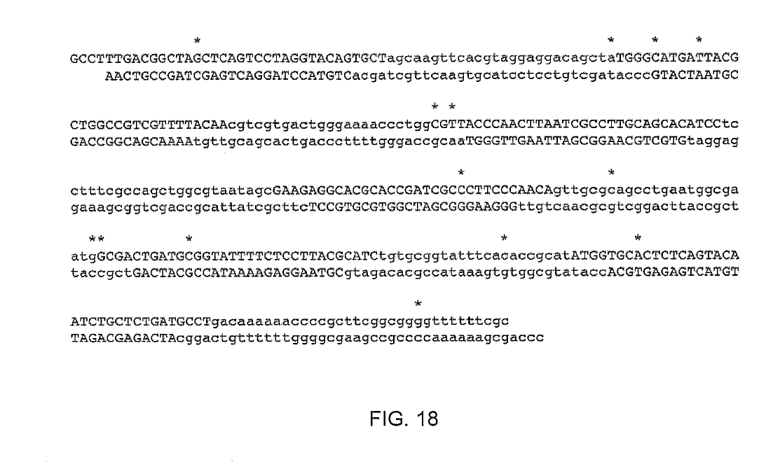

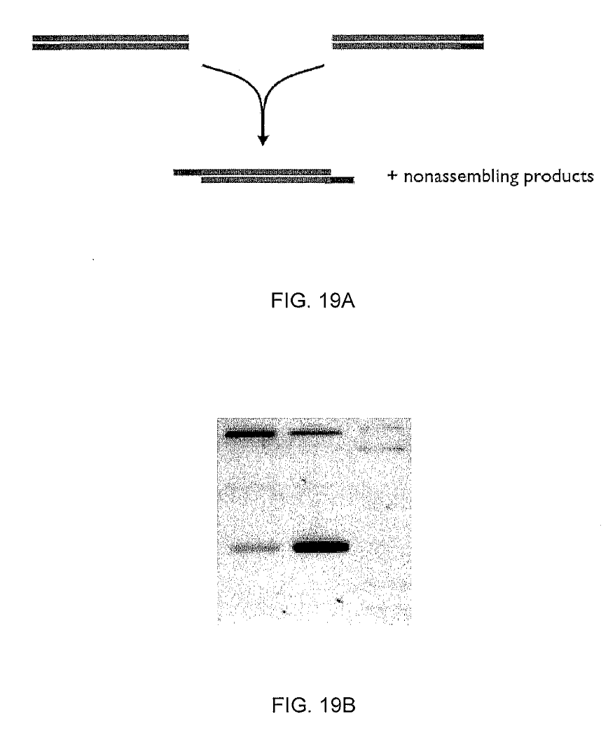

In one embodiment, the bytes for use in the method of the present invention may be produced by direct ligation of synthetic oligos (FIG. 18, Example 8). In another embodiment, the bytes may be produced by heteroduplexing a pair of linear PCR products (FIGS. 19A-B, Example 9).

Direct annealing of synthesized oligonucleotides is suitable for short linkers which are difficult to purify, and for the anchors and terminators which have long sticky extensions which are difficult to produce enzymatically.

Example 4 demonstrates the method of producing multi-component DNA constructs (i.e., an octomer) using the nucleic acid components. Briefly, the anchor is prepared and bound to the support. Binding of the first byte to the anchor is achieved by incubation, followed by washing to remove any unbound first byte (cycle 1, FIG. 1). The chain is constrained to grow in only one direction, namely away from the anchor. A second byte is ligated to the free terminal sequence or sticky end of the first byte by repeating the incubation and wash steps (cycle 2, FIG. 1). A third byte is ligated to the free terminal sequence or sticky end of the second byte by repeating the incubation and wash steps (cycle 3, FIG. 1). As desired, additional bytes can be annealed and linked to the growing chain in the same manner until the desired DNA plasmid or construct has been generated. The final DNA plasmid or construct is then eluted from the support.

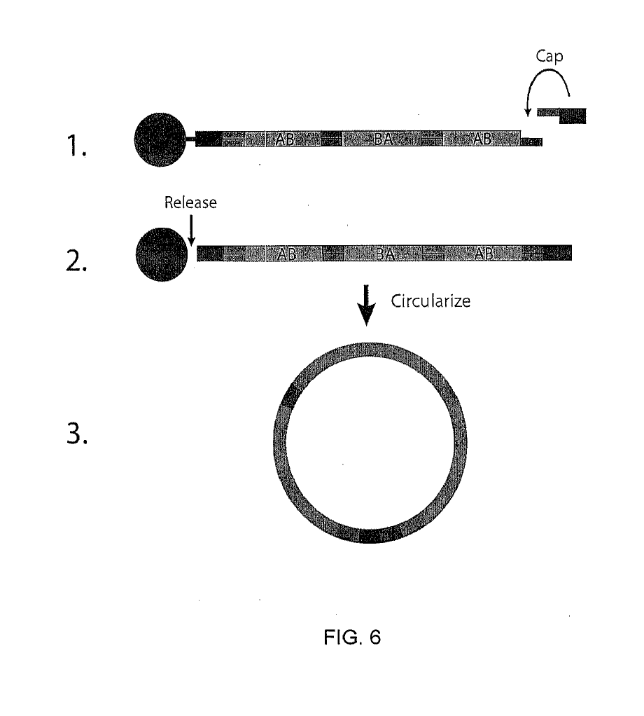

If the final added nucleic acid component is a terminator designed to anneal to the anchor sequence, the eluted construct spontaneously circularizes to form a transformable plasmid (FIG. 6). In one embodiment, the terminator comprises a poly-dT end cap at its 5′ end which anneals to a complementary poly-dA anchor. The binding is strong enough that ligation is not required prior to transformation. After elution from the beads, the resulting circularized DNA may be transformed into cells or further processed.

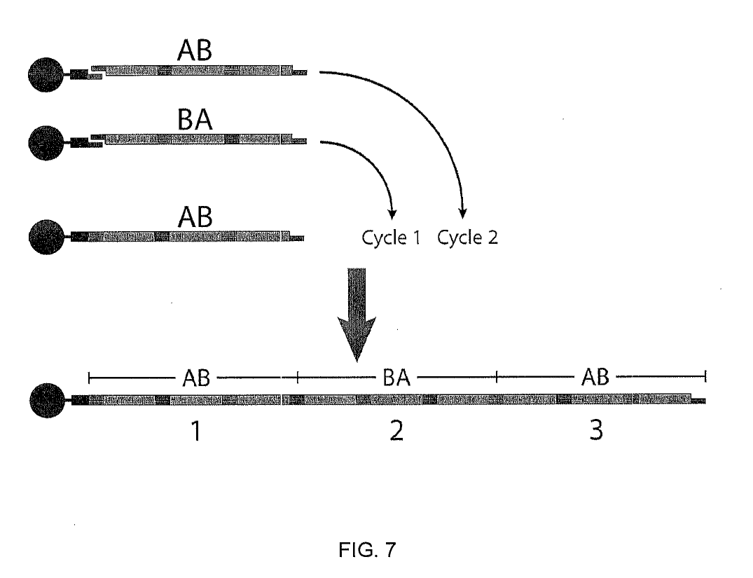

In one embodiment, the method comprises hierarchical parallel assembly of the nucleic acid components on the support to generate the desired DNA plasmid or construct. As shown in FIG. 7, multiple parallel assemblies are conducted as described above, beginning with A anchors and B anchors, and ending with the opposite type. A Type IIs restriction site in the anchor allows release of a construct by enzymatic digestion, leaving a ligatable A (or alternatively B) end, with the opposite type at the other end. The released multipart construct is thus also an AB (or alternatively BA) byte which can be used in further assemblies without cloning. The multipart constructs may then be attached to anchors and assembled using the method previously described. This parallel construction method may be particularly amenable to automation, with either conventional lab-scale robotics or through lab-on-a-chip type microfluidic approaches.

The method of the present invention has been described as comprising a series of steps, each adding a single part to the growing chain, where the parts come from a purified solution. However, it will be appreciated by those skilled in the art that the method of the present invention can be easily extended to allow library construction. By mixing together several parts of the same type (e.g., the AB byte), one or another will be added at that stage, resulting in a library whose components have the same length and a controlled distribution of desired parts at every stage. These libraries present a substantial improvement over classical methods of gene shuffling.

In the method, the nucleic acid components are assembled in a single defined direction, resulting in all the nucleic acid components of the plasmid or construct ending on the same strand of DNA. If this is not desired, a direction reversing linker can be used; for example, a part with A and B sticky ends could bind to both the B′ end of a standard AB byte and the A′ end of a standard BA byte, reversing the usual orientation between the standard bytes. After a series of construction with the reversed bytes, another direction reversing linker would be required to complete a circularizable construct.

In one embodiment, the invention provides a kit for assembly of a DNA construct comprising a plurality of first form and second form nucleic acid components, wherein the first and second forms of nucleic acid component comprises sticky ends such that each form cannot link to itself but can link to each other to form the DNA construct comprising an alternating head to tail sequence.

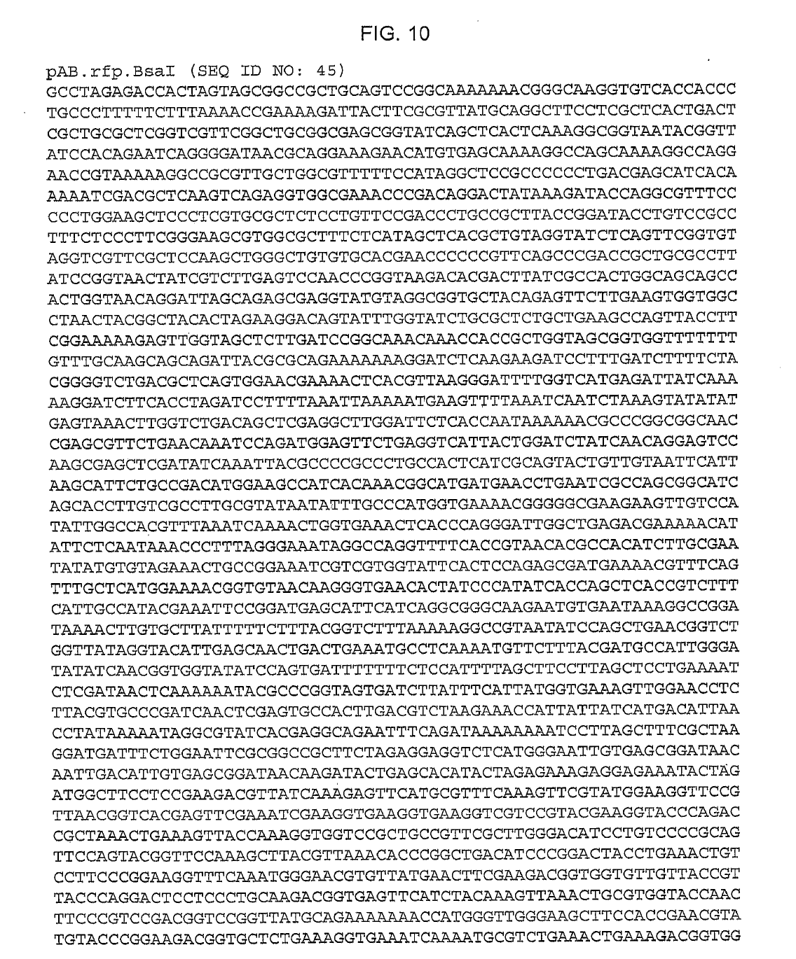

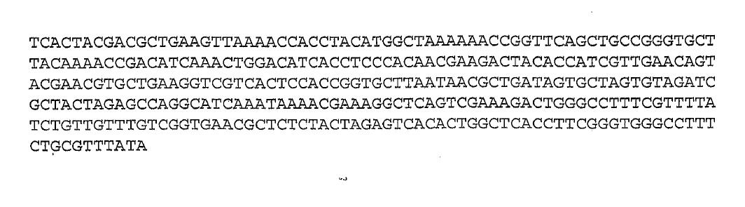

As set out in Table 2, an exemplary kit includes complementary nucleic acid components, allowing construction of a wide variety of DNA constructs, and using the categorization of the nucleic acid components described above. The kit includes, but is not limited to, replication origins, antibiotic resistance cassettes, controllers, reporters in the forms of visible pigments or fluorescent proteins, constitutive and regulated promoters, operators, linkers, anchors, terminators, and plasmids. Exemplary DNA constructs are set out in FIGS. 10-15 and as set out in SEQ ID NOS: 45-50.

TABLE 3
Contents of Kit for Assembly of Desired Nucleic Acid Molecules

| | | End | | Se- | |
|---|---|---|---|---|---|
| | End Type | generator | Made | quenced | Tested |
| Rep Origins | | | | | |
| pMB1 (high-copy) | AB & BA | Bsal | | | |
| p15A (medium copy) | AB & BA | Bsal | | | |
| pSC101 (low copy) | AB & BA | Bsal | | | |
| Antibiotic resistance | | | | | |
| AmpR | AB & BA | Bsal | + | + | + |
| ChlrR | AB & BA | Bsal | + | + | + |
| KanR | AB & BA | Bsal | + | + | + |
| TetR | AB & BA | Bsal | + | + | + |
| Controllers | | | | | |
| Lacl repressor | AB | Bsal | + | + | |
| λC1 repressor | AB | Bsal | + | + | |
| AraC repressor | AB | Bsal | + | + | |
| TetRo repressor | AB | Bsal | + | + | |
| Reporters | | | | | |
| GFP | AB | Bsal | | | |
| RFP | AB | Bsal | + | + | |
| CFP | AB | Bsal | | | |
| Cambridge colours | | | | | |
| (Green, Orange, | | | | | |
| Violet) | | | | | |
| CrtB, E, I, Y, Z | AB | Bsal | +/− | +/− | |
| VioA, B, C, D, E | AB | Bsal | +/− | +/− | |

| | End Type | Made | Tested |
|---|---|---|---|
| Constitutive promoters (Pr + Rbs) | BA | + | |
| Relative strength: 1000 | | | |
| 562 | | | |
| 248 | | | |
| 150 | | | |
| 64 | | | |
| Regulated Promoters (O + Pr + Rbs) | BA | + | |
| AraC | | | |
| λ Cl | | | |
| Lacl | | | |
| TetRo | | | |
| mRNA terminator (trp attr) | BA | + | |
| negative control | | | |
| mRNA Lkr (stp-Rbs) | BA | + | |
| negative control | | | |
| End inverter (A to B) | AB | + | |
| (B to A) | BA | + | |

Table 3 (Example 5) sets out exemplary sequences for the above nucleic acid components, including the sticky ends. The first four bases comprise the 5′ overhang on the top strand, while the last four bases comprise the reverse complement of the 5′ overhang on the unwritten bottom strand. The sequences are designated by their part numbers in accordance with the Registry of Standard Biological Parts. Modifications are indicated to particular sequences where applicable. In the case that no sequence is provided for a part, it will be understood that the sequence comprises the native sequence with attached sticky ends. In one embodiment, the nucleic acid component comprises a sequence as set forth in any one of SEQ ID NOS: 1 to 40.

In one embodiment, the invention provides a composition comprising one or more nucleic acid components as set forth in any one of SEQ ID NOS: 1-40 and 45-50.

In one embodiment, the invention provides a vector comprising a sequence as set forth in any one of SEQ ID NOS: 45-50.

Exemplary embodiments of the present invention are described in the following Examples, which are set forth to aid in the understanding of the invention, and should not be construed to limit in any way the scope of the invention as defined in the claims which follow thereafter.

### Example 1

### Preparation and Production of DNA Molecules

All parts are initially created by PCR amplification of the target sequence using forward and reverse primers that appends (for example) BsaI recognition sites to either end in an orientation that falls outside of the desired sequence. A buffer of 12 bases is added to the 5′ end of each primer to assure efficient cleavage by the restriction endonuclease. Cleavage with BsaI results in A/B′ overhangs in the case of an AB module and a B/A′ overhangs in the case of a BA module. BA Primer sequences for the Kanamycin resistance cassette (iGEM parts registry BBa_p1003) are shown below:

Forward (A overhang)
(SEQ ID NO: 41)
5′GCCGCTTCTAGAGGTCTCATGGGCTGATCCTTCAACTCAGCAAAAGTT
C
Reverse (B overhang)
(SEQ ID NO: 42)
5′GCCGCTTCTAGAGGTCTCAGCCTCTGATCCTTCAACTCAGCAAAAGTT
C

BsaI sites are underlined. Overhang sequences are highlighted in bold. Sequences to the right of the overhangs correspond to sequences at the boundaries of BBa_p1003.

Upon cleavage with BsaI, modules are introduced into one of two specially created cloning vectors (pAB or pBA). pAB is the recipient of AB-type modules whereas pBA is the recipient of BA-type modules. The sequences of both plasmids are identical to pSB1C3 from the iGEM parts registry which carries the cassette for red fluorescent protein (BBa_I13521) with the exception of the BsaI recognition and overhangs underlined below:

pAB
(SEQ ID NO: 43)
GAATTCGCGGCCGCTTCTAGAGGTCTCATGGG[BBa_I13521]GCCTAGAGACCACTAGTTGC
GGCCGCTGCAG
pBA
(SEQ ID NO: 44)
GAATTCGCGGCCGCTTCTAGAGGTCTCAGCCT[BBa_I13521]TGGGAGAGACCACTAGTTGC
GGCCGCTGCAG

Cleaved modules are ligated into the appropriate plasmid that has been cut with BsaI. Candidate colonies for inserts appear white whereas red/pink colonies denote uncut host plasmid contaminant, or, the reintroduction of the RFP cassette still present in the reaction. The overhangs at the ends of either plasmid are refractory to ligation, therefore backbone recircularization is extremely rare, the end result is that white colonies almost always contain inserts. Upon verification of candidates based on the size of the module released by BsaI cleavage of miniprep DNA all modules are sequenced using the Registry primers Vf and Yr. DNA plasmids useful in the method of the present invention are set out in FIGS. 10-15 and as set out in SEQ ID NOS: 45-50.

### Example 2

### Preparation of Modules for Assembly

Modules that are used for solid support assembly are first PCR amplified from their plasmids using in an optimized PCR reaction (below) using universal primers whose sequences are derived from entirely from the pSB1C3 backbone (shown below):

Forward (BBy.Vf)
(SEQ ID NO: 51)
GATTTCTGGAATTCGCGGCCGCTTCTAGAG
Reverse (BBy.Vr)
(SEQ ID NO: 52)
CGGACTGCAGCGGCCGCTACTAGTA

Each primer has been selected to initiate synthesis ˜150 bp away from its module boundary, a distance that has been determined to be necessary and sufficient to gauge the efficiency of BsaI by gel electrophoresis. This is an important consideration in quality control since the presence of partially cut modules significantly reduces the efficiency of solid support assembly. The optimized cleavage reaction is: 10 pmoles of PCR product in 50 μL 1×NEB buffer 4, with BSA, +20 units BsaI, incubated at 37° C. for 3 hours.

Modules are then purified from their cleaved flanks and enzyme by weak anion exchange HPLC chromatography using an DNA-NPR solid pore DEAE column (TSK-GEL; resin #18249, column #R0028) at a flow rate of 0.5 mL/min in 50 mM Tris-HCl (pH 8), 1 mM EDTA) throughout, and over a NaCl gradient of 0-1M. A sample profile of the Kan module is shown below.

In practice, this method is preferred over gel purification in terms of capacity (100 μg), yield (>90%) and speed (˜15 min/run), while minimizing exposure to UV and contaminants. Excess NaCl is then removed from the collected module peak (˜0.6 M) using the Qiagen™ “quick cleanup kit” followed by resuspension of the module in 10 mM Tris, 1 mM EDTA, pH 8.0 at final concentration that has been optimized for the assembly reaction (1 pMole/10 μL).

### Example 3

### Module PCR Amplification

PCR Optimization Using Universal Primers:

Preheat the PCR machine using the following program:
1. 3 minutes at 94° C.
2. 45 seconds at 94° C.
3. 30 seconds at 62° C.
4. 90 seconds at 72° C.
5. Cycle steps 2-4 25 times
6. 10 minutes at 72° C.

Prepare the PCR Reactions. It does not matter what order the reagents are added as long as the enzyme is added last. The PCRs are kept on ice until the PCR machine is ready. Recipe per 1 PCR Reaction:
1.0 μL template @ 1 μg/ml; 1 ng total)
1.0 μL 10 mM dNTPs
2.0 μL 50 mM MgCl₂
2.5 μL BBy_Vf (1/10 dilution from primer stock @100 nM/mL)
2.5 μL BBy_Vr (1/10 dilution from primer stock @100 nM/mL)
5.0 μl, 10× Taq Buffer
35.5 μL MilliQ H₂O
0.5 λL Taq Polymerase
TOTAL of 504
Add 50 μL of mineral oil and run the reactions.

### Example 4

### Construction of Octomer

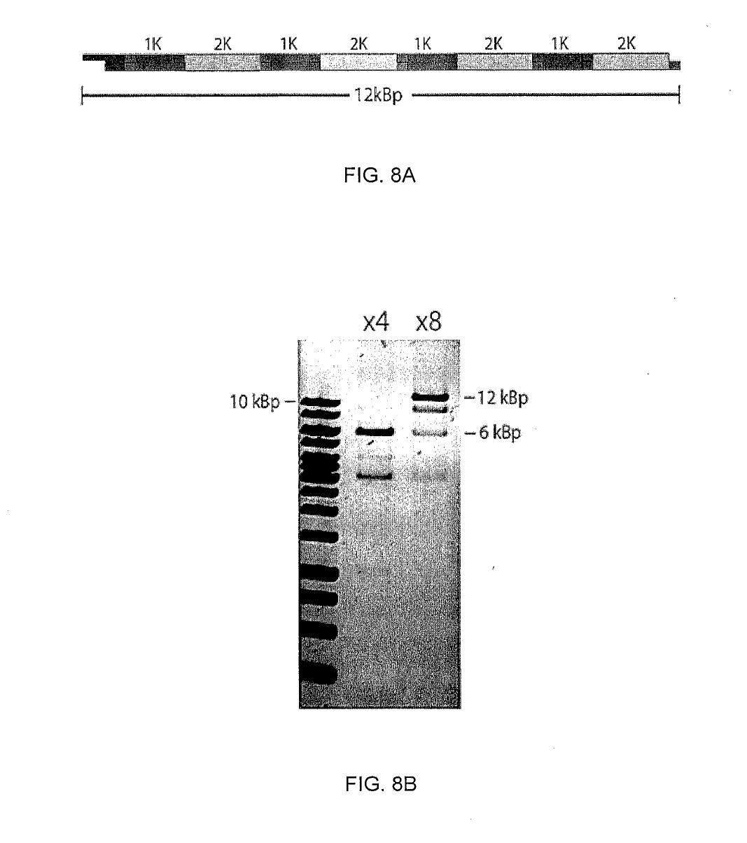

The method incorporating bytes, anchors and terminators was demonstrated by construction of an octomer (FIG. 8A). The anchor is prepared by mixing 4 μmol of the initial 0.9 kb AB byte with 50 μmol of A anchor, with 10 Quick Ligase™ (New England Biolabs, Ipswich, Mass.) in 40 μL Quick Ligase™ buffer, incubating for 5 minutes at room temperature, followed by heat inactivation at 65° C. for 10 minutes. To bind to the oligo-dT paramagnetic beads (New England Biolabs, #S1419S), the beads are resuspended by shaking and swirling and washed twice with 50 μl TE buffer. During a wash, a magnet is applied to the side of the tube to pellet the beads and allow for convenient solution change. 4 μl of anchor prepared with 16 μl of TE buffer is added at room temperature. The tube is flicked and inverted for 30 seconds, then washed twice.

The addition of bytes is achieved by resuspending the beads with 4 μl of BA byte (0.4 μmol) in 20 μl ligase buffer with ligase, and incubating for five minutes at room temperature with gentle mixing, then washing twice as described above. Additional bytes can be added as desired, for example, up to eight in total for an octamer.

The final construct is eluted by mixing the bead pellet with 20 μl of elution buffer and heating to 70° C. The beads are pelleted and the supernatant is removed. The final construct remains in the supernatant, and can be visualized by gel electrophoresis (FIG. 8B). While most of the material collects in the band of the desired size, several bands of truncated products are visible.

If the final added part is a terminator designed to anneal to the anchor sequence, the eluted construct spontaneously circularizes to form a transformable plasmid (FIG. 6). In one embodiment, the terminator comprises a poly-dT end cap at its 5′ end. The poly-dT cap anneals to a poly-dA anchor. The binding is strong enough that ligation is not required for transformation. After elution from the beads, the resulting circularized DNA may be transformed into cells or further processed.

### Example 5

### Sequences for Bytes (Table 4)

TABLE 4
Sequences for Bytes (SEQ ID NOS: 1-40)

| | | | | Modifi- |
|---|---|---|---|---|
| | | | DNA | ca- |
| | | | or | tions |
| | | | Se- | from |
| | Num- | | quence | Original |
| Part | ber | Sequence | Source | Sequence |
| pMB1 | | native sequence | | |
| | | (SEQ ID NO: 1) | | |
| P1SA | | native sequence | | |
| | | (SEQ ID NO: 2) | | |
| pSC101 | | native sequence | | |
| | | (SEQ ID NO: 3) | | |
| AmpR | | native sequence | | |
| | | (SEQ ID NO: 4) | | |
| ChlrR | | native sequence | | |
| | | (SEQ ID NO: 5) | | |
| KanR | | native sequence | | |
| | | (SEQ ID NO: 6) | | |
| TetR | | native sequence | | |
| | | (SEQ ID NO: 7) | | |
| LacI | | TGGGtccagtaacgttatacgatgtcgcagagtatgccggtgtctcttatcagaccgtttcccgcgtggtgaac | Bba_ | N and C |
| | | caggccagccacgtttctgcgaaaacgcgggaaaaagtggaagcggcgatggcggagctgaattacattcccaa | C0012 | terminal |
| | | ccgcgtggcacaacaactggcgggcaaacagtcgttgctgattggcgttgccacctccagtctggccctgcacg | | con- |
| | | cgccgtcgcaaattgtcgcggcgattaaatctcgcgccgatcaactgggtgccagcgtggtggtgtcgatggta | | verted |
| | | gaacgaagcggcgtcgaagcctgtaaagcggcggtgcacaatcttctcgcgcaacgcgtcagtgggctgatcat | | to wt |
| | | taactatccgctggatgaccaggatgccattgctgtggaagctgcctgcactaatgttccggcgttatttcttg | | version |
| | | atgtctctgaccagacacccatcaacagtattattttctcccatgaagacggtacgcgactgggcgtggagcat | | then |
| | | ctggtcgcattgggtcaccagcaaatcgcgctgttagcgggcccattaagttctgtctcggcgcgtctgcgtct | | placed |
| | | ggctggctggcataaatatctcactcgcaatcaaattcagccgatagcggaacgggaaggcgactggagtgcca | | in byte |
| | | tgtccggttttcaacaaaccatgcaaatgctgaatgagggcatcgttcccactgcgatgctggttgccaacgat | | format. |
| | | cagatggcgctgggcgcaatgcgcgccattaccgagtccgggctgcgcgttggtgcggatatctcggtagtggg | | |
| | | atacgacgataccgaagacagctcatgttatatcccgccgtTaaccaccatcaaacaggattttcgcctgctgg | | |
| | | ggcaaaccagcgtggaccgcttgctgcaactctctcagggccaggcggtgaagggcaatcagctgttgcccgtc | | |
| | | tcactggtgaaaagaaaaaccaccctggcgcccaatacgcaaaccgcctctccccgcgcgttggccgattcatt | | |
| | | aatgcagctggcacgacaggtttcccgactggaaagcgggGCCT | | |
| | | (SEQ ID NO: 8) | | |
| lambda | | TGGGTacaaaaaagaaaccattaacacaagagcagcttgaggacgcacgtcgccttaaagcaatttatgaaaaa | BBa_ | C |
| C1 | | aagaaaaatgaacttggcttatcccaggaatctgtcgcagacaagatggggatggggcagtcaggcgttggtgc | C0051 | terminal |
| | | tttatttaatggcatcaatgcattaaatgcttataacgccgcattgcttgcaaaaattctcaaagttagcgttg | | sequence |
| | | aagaatttagcccttcaatcgccagagaaatctacgagatgtatgaagcggttagtatgcagccgtcacttaga | | con- |
| | | agtgagtatgagtaccctgttttttacatgttcaggcagggatgttctcacctgagcttagaacctttaccaaa | | verted |
| | | ggtgatgcggagagatgggtaagcacaaccaaaaaagccagtgattctgcattctggcttgaggttgaaggtaa | | to wt |
| | | ttccatgaccgcaccaacaggctccaagccaagctttcctgacggaatgttaattctcgttgaccctgagcagg | | version |
| | | ctgttgagccaggtgatttctgcatagccagacttgggggtgatgagtttaccttcaagaaactgatcagggat | | then |
| | | agcggtcaggtgtttttacaaccactaaacccacagtacccaatgatcccatgcaatgagagttgttccgttgt | | placed |
| | | ggggaaagttatcgctagtcagtggcctgaagagacgtttGCCT | | in byte |
| | | (SEQ ID NO: 9) | | format. |
| AraC | | TGGGtgaagcgcaaaatgatcccctgctgccgggatactcgtttaacgcccatctggtggcgggtttaacgccg | BBa_ | C |
| | | attgaggccaatggttatctcgatttttttatcgaccgaccgctgggaatgaaaggttatattctcaatctcac | C0080 | terminal |
| | | cattcgcggtcagggggtggtgaaaaatcagggacgagaatttgtctgccgaccgggtgatattttgctgttcc | | sequence |
| | | cgccaggagagattcatcactacggtcgtcatccggaggctcgcgaatggtatcaccagtgggtttactttcgt | | con- |
| | | ccgcgcgcctactggcatgaatggcttaactggccgtcaatatttgccaatacgggtttctttcgcccggatga | | verted |
| | | agcgcaccagccgcatttcatgcgacctgttgggcaaatcattaacgccgggcaaggggaagggcgctattcgg | | to wt |
| | | agctgctggcgataaatctgcttgagcaattgttactgcggcgcatggaagcgattaacgagtcgctccatcca | | version |
| | | ccgatggataatcgggtacgcgaggcttgtcagtacatcagcgatcacctggcagacagcaattttgatatcgc | | then |
| | | cagcgtcgcacagcatgtttgcctgtcgccgtcgcgtctgtcacatcttttccgccagcagttagggattagcg | | placed |
| | | tcttaagctggcgcgaggaccaacgcatcagccaggcgaagctgcttttgagcactacccggatgcctatcgcc | | in byte |
| | | accgtcggtcgcaatgttggttttgacgatcaactctatttctcgcgagtatttaaaaaatgcaccggggccag | | format. |
| | | cccgagcgagttccgtgccggttgtgaagaaaaagtgaatgatgtagccgtcaagttgGCCT | | |
| | | (SEQ ID NO: 10) | | |
| TetRo | | TGGGtccagtaacgttatacgatgtcgcagagtatgccggtgtctcttatcagaccgtttcccgcgtggtgaac | Bba_ | C |
| | | caggccagccacgtttctgcgaaaacgcgggaaaaagtggaagcggcgatggcggagctgaattacattcccaa | C0040 | terminal |
| | | cccgcgtggacaacaactggcgggcaaacagtcgttgctgattggcgttgccacctccagtctggccctgcacg | | con- |
| | | cgccgtcgcaaattgtcgcggcgattaaatctcgcgccgatcaactgggtgccagcgtggtggtgtcgatggta | | verted |
| | | gaacgaagcggcgtcgaagcctgtaaagcggcggtgcacaatcttctcgcgcaacgcgtcagtgggctgatcat | | to wt |
| | | taactatccgctggatgaccaggatgccattgctgtggaagctgctgccactaatgttccggcgttatttcttg | | version |
| | | atgtctctgaccagacacccatcaacagtattattttctcccatgaagacggtacgcgactgggcgtggagcat | | then |
| | | ctggtcgcattgggtcaccagcaaatcgcgctgttagcgggcccattaagttctgtctcggcgcgtctgcgtct | | placed |
| | | ggctggctggcataaatatctcactcgcaatcaaattcagccgatagcggaacgggaaggcgactggagtgcca | | in byte |
| | | tgtccggttttcaacaaaccatgcaaatgctgaatgagggcatcgttcccactgcgatgctggttgccaacgat | | format. |
| | | cagatggcgctgggcgcaatgcgcgccattaccgagtccgggctgcgcgttggtgcggatatctcggtagtggg | | |
| | | atacgacgataccgaagacagctcatgttatatcccgccgtTaaccaccatcaaacaggattttcgcctgctgg | | |
| | | ggcaaaccagcgtggaccgcttgctgcaactctctcagggccaggcggtgaagggcaatcagctgttgcccgtc | | |
| | | tcactggtgaaaagaaaaaccaccctggcgcccaatacgcaaaccgcctctccccgcgcgttggccgattcatt | | |
| | | aatgcagctggcacgacaggtttcccgactggaaagcgggGCCT | | |
| | | (SEQ ID NO: 11) | | |
| GFP | | GAATTCGCGGCCGCTTCTAGAGGTCTCATGGGTaaaggagaagaacttttcactggagttgtcccaattcttgt | BBa_ | |
| | | tgaattagatggtgatgttaatgggcacaaattttctgtcagtggagagggtgaaggtgatgcaacatacggaa | I13522 | |
| | | aacttacccttaaatttatttgcactactggaaaactacctgttccatggccaacacttgtcactactttcggt | | |
| | | tatggtgttcaatgctttgcgagatacccagatcatatgaaacagcatgactttttcaagagtgccatgcccga | | |
| | | aggttatgtacaggaaagaactatatttttcaaagatgacgggaactacaagacacgtgctgaagtcaagtttg | | |
| | | aaggtgatacccttgttaatagaatcgagttaaaaggtattgattttaaagaagatggaaacattcttggacac | | |
| | | aaattggaatacaactataactcacacaatgtatacatcatggcagacaaacaaaagaatggaatcaaagttaa | | |
| | | cttcaaaattagacacaacattgaagatggaagcgttcaactagcagaccattatcaacaaaatactccaattg | | |
| | | gcgatggccctgtccttttaccagacaaccattacctgtccacacaatctgccctttcgaaagatcccaacgaa | | |
| | | aagagagaccacatggtccttcttgagtttgtaacagctgctgggattacacatggcatggatgaactatacaa | | |
| | | aGCCTAGAGACCACTAGTTGCGGCCGCTGCAG | | |
| | | (SEQ ID NO: 12) | | |
| RFP | | TGGGTtcctccgaagacgttatcaaagagttcatgcgtttcaaagttcgtatggaaggttccgttaacggtcac | BBa_ | |
| | | gagttcgaaatcgaaggtgaaggtgaaggtcgtccgtacgaaggtacccagaccgctaaactgaaagttaccaa | I13521 | |
| | | aggtggtccgctgccgttcgcttgggacatcctgtccccgcagttccagtacggttccaaagcttacgttaaac | | |
| | | acccggctgacatcccggactacctgaaactgtccttcccggaaggtttcaaatgggaacgtgttatgaacttc | | |
| | | gaagacggtggtgttgttaccgttacccaggactcctccctgcaagacggtgagttcatctacaaagttaaact | | |
| | | gcgtggtaccaacttcccgtccgacggtccggttatgcagaaaaaaaccatgggttgggaagcttccaccgaac | | |
| | | gtatgtacccggaagacggtgctctgaaaggtgaaatcaaaatgcgtctgaaactgaaagacggtggtcactac | | |
| | | gacgctgaagttaaaaccacctacatggctaaaaaaccggttcagctgccgggtgcttacaaaaccgacatcaa | | |
| | | actggacatcacctcccacaacgaagactacaccatcgttgaacagtacgaacgtgctgaaggtcgtcactcca | | |
| | | ccggtgcttaataa | | |
| | | (SEQ ID NO: 13) | | |
| CFP | | native sequence | | |
| | | (SEQ ID NO: 14) | | |
| CrtB | | GAATTCGCGGCCGCTTCTAGAGGTCTCATGGGTaatccgtcgttactcaatcatgcggtcgaaacgatggcagt | K274100 | |
| | | tggctcgaaaagttttgcgacagcctcaaagttatttgatgcaaaaacccggcgcagcgtactgatgctctacg | (ind. | |
| | | cctggtgccgccattgtgacgatgttattgacgatcagacgctgggctttcaggcccggcagcctgccttacaa | K118002) | |
| | | acgcccgaacaacgtctgatgcaacttgagatgaaaacgcgccaggcctatgcaggatcgcagatgcacgaacc | | |
| | | ggcgtttgcggcttttcaggaagtggctatggctcatgatatcgccccggcttacgcgtttgatcatctggaag | | |
| | | gcttcgccatggatgtacgcgaagcgcaatacagccaactggatgatacgctgcgctattgctatcacgttgca | | |
| | | ggcgttgtcggcttgatgatggcgcaaatcatgggcgtgcgggataacgccacgctggaccgcgcctgtgacct | | |
| | | tgggctggcatttcagttgaccaatattgctcgcgatattgtggacgatgcgcatgcgggccgctgttatctgc | | |
| | | cggcaagctggctggagcatgaaggtctgaacaaagagaattatgcggcacctgaaaaccgtcaggcgctgagc | | |
| | | cgtatcgcccgtcgtttggtgcaggaagcagaaccttactatttgtctgccacagccggcctggcagggttgcc | | |
| | | cctgcgttccgcctgggcaatcgctacggcgaagcaggtttaccggaaaataggtgtcaaagttgaacaggccg | | |
| | | gtcagcaagcctgggatcagcggcagtcaacgaccacgcccgaaaaattaacgctgctgctggccgcctctggt | | |
| | | caggcccttacttcccggatgcgggctcatcctccccgccctgcgcatctctggcagcgcccgGCCTAGAGACC | | |
| | | ACTAGTTGCGGCCGCTGCAG | | |
| | | (SEQ ID NO: 15) | | |
| CrtE | | GAATTCGCGGCCGCTTCTAGAGGTCTCATGGGTgtctgcgcaaaaaaacacgttcatctcactcgcgatgctgc | K274100 | |
| | | ggagcagttactggctcatattgatcgacgccttgatcagttattgcccgtggagggagaacgggatgttgtgg | (ind. | |
| | | gtgccgcgatgcgtgaaggtgcgctggcaccgggaaaacgtattcgccccatgttgctgttgctgaccgcccgc | I742515) | |
| | | gatctgggttgcgctgtcagccatgacggattactggatttggcctgtgcggtggaaatggtccacgcggcttc | | |
| | | gctgatccttgacgatatgccctgcatggacgatgcgaagctgcggcgcggacgccctaccattcattctcatt | | |
| | | acggagagcatgtggcaatactggcggcggttgccttgctgagtaaagcctttggcgtaattgccgatgcagat | | |
| | | cggctcacgccgctggcaaaaaatcgggcggtttctgaactgtcaaacgccatcggcatgcaaggattggttca | | |
| | | gggtcagttcaaggatctgtctgaaggggataagccgcgcagcgctgaagctattttgatgacgaatcacttta | | |
| | | aaaccagcacgctgttttgtgcctccatgcagatggcctcgattgttgcgaatgcctccagcgaagcgcgtgat | | |
| | | tgcctgcatcgtttttcacttgatcttggtcaggcatttcaactgctggacgatttgaccgatggcatgaccga | | |
| | | caccggtaaggatagcaatcaggacgccggtaaatcgacgctggtcaatctgttaggcccgagggcggttgaag | | |
| | | aacgtctgagacaacatcttcagcttgccagtgagcatctctctgcggcctgccaacacgggcacgccactcaa | | |
| | | cattttattcaggcctggtttgacaaaaaactcgctgccgtcagGCCTAGAGACCACTAGTTGCGGCCGCTGCA | | |
| | | G | | |
| | | (SEQ ID NO: 16) | | |
| CrtI | | GAATTCGCGGCCGCTTCTAGAGGTCTCATGGGTccaactacggtaattggtgcaggcttcggtggcctggcact | K274100 | |
| | | ggcaattcgtctacaagctgcggggatccccgtcttactgcttgaacaacgtgataaacccggcggtcgggctt | (ind. | |
| | | atgtctacgaggatcaggggtttacctttgatgcaggcccgacggttatcaccgatcccagtgccattgaagaa | K118003) | |
| | | ctgtttgcactggcaggaaaacagttaaaagagtatgtcgaactgctgccggttacgccgttttaccgcctgtg | | |
| | | ttgggagtcagggaaggtctttaattacgataacgatcaaacccggctcgaagcgcagattcagcagtttaatc | | |
| | | cccgcgatgtcgaaggttatcgtcagtttctggactattcacgcgcggtgtttaaagaaggctatctaaagctc | | |
| | | ggtactgtcccttttttatcgttcagagacatgcttcgcgccgcacctcaactggcgaaactgcaagcatggag | | |
| | | aagcgtttacagtaaggttgccagttacatcgaagatgaacatctgcgccaggcgttttctttccactcgctgt | | |
| | | tggtgggcggcaatcccttcgccacctcatccatttatacgttgatacacgcgctggagcgtgagtggggcgtc | | |
| | | tggtttccgcgtggcggcaccggcgcattagttcaggggatgataaagctgtttcaggatctgggtggcgaagt | | |
| | | cgtgttaaacgccagagtcagccatatggaaacgacaggaaacaagattgaagccgtgcatttagaggacggtc | | |
| | | gcaggttcctgacgcaagccgtcgcgtcaaatgcagatgtggttcatacctatcgcgacctgttaagccagcac | | |
| | | cctgccgcggttaagcagtccaacaaactgcaaactaagcgcatgagtaactctctgtttgtgctctattttgg | | |
| | | tttgaatcaccatcatgatcagctcgcgcatcacacggtttgtttcggcccgcgttaccgcgagctgattgacg | | |
| | | aaatttttaatcatgatggcctcgcagaggacttctcactttatctgcacgcgccctgtgtcacggattcgtca | | |
| | | ctggcgcctgaaggttgcggcagttactatgtgttggcgccggtgccgcatttaggcaccgcgaacctcgactg | | |
| | | gacggttgaggggccaaaactacgcgaccgtatttttgcgtaccttgagcagcattacatgcctggcttacgga | | |
| | | gtcagctggtcacgcaccggatgtttacgccgtttgattttcgcgaccagcttaatgcctatcatggctcagcc | | |
| | | ttttctgtggagcccgttcttacccagagcgcctggtttcggccgcataaccgcgataaaaccattactaatct | | |
| | | ctacctggtcggcgcaggcacgcatcccggcgcaggcattcctggcgtcatcggctcggcaaaagcgacagcag | | |
| | | gtttgatgctggaggatctgGCCTAGAGACCACTAGTTGCGGCCGCTGCAG | | |
| | | (SEQ ID NO: 17) | | |
| CrtY | | GAATTCGCGGCCGCTTCTAGAGGTCTCATGGGTccgcattatgatctgattctcgtgggggctggactcgcgaa | K274200 | |
| | | tggccttatcgccctgcgtcttcagcagcagcaacctgatatgcgtattttgcttatcgacgccgcaccccagg | (ind. | |
| | | cgggcgggaatcatacgtggtcatttcaccacgatgatttgactgagagccaacatcgttggatagctccgctg | K118008) | |
| | | gtggttcatcactggcccgactatcaggtacgctttcccacacgccgtcgtaagctgaacagcggctacttttg | | |
| | | tattacttctcagcgtttcgctgaggttttacagcgacagtttggcccgcacttgtggatggataccgcggtcg | | |
| | | cagaggttaatgcggaatctgttcggttgaaaaagggtcaggttatcggtgcccgcgcggtgattgacgggcgg | | |
| | | ggttatgcggcaaattcagcactgagcgtgggcttccaggcgtttattggccaggaatggcgattgagccaccc | | |
| | | gcatggtttatcgtctcccattatcatggatgccacggtcgatcagcaaaatggttatcgcttcgtgtacagcc | | |
| | | tgccgctctcgccgaccagattgttaattgaagacacgcactatattgataatgcgacattagatcctgaatgc | | |
| | | gcgcggcaaaatatttgcgactatgccgcgcaacagggttggcagcttcagacactgctgcgagaagaacaggg | | |
| | | cgccttacccattactctgtcgggcaatgccgacgcattctggcagcagcgccccctggcctgtagtggattgc | | |
| | | acgtgccggtctgttccatcctaccaccggctattcactgccgctgggttgccgtggccgaccgcctgagtgca | | |
| | | cttgatgtctttacgtcggcctcaattcaccatgccattacgcattttgcccgcgagcgctggcagcagcaggg | | |
| | | ctttttccgcatgctgaatcgcatgctgtttttagccggacccgccgattcacgctggcgggttatgcagcgtt | | |
| | | tttatggtttacctgaagatttaattgcccgtttttatgcgggaaaactcacgctgaccgatcggctacgtatt | | |
| | | ctgagcggcaagccgcctgttccggtattagcagcattgcaagccattatgacgactcatGCCTAGAGACCACT | | |
| | | GATTGCGGCCGCTGCAG | | |
| | | (SEQ ID NO: 18) | | |
| CrtZ | | GAATTCGCGGCCGCTTCTAGAGGTCTCATGGGTtggatttggaatgccctgatcgttttcgttaccgtgattgg | I742157 | |
| | | catggaagtgattgctgcactggcacacaaatacatcatgcacggctggggttggggatggcatctttcacatc | | |
| | | atgaaccgcgtaaaggtgcgtttgaagttaacgatctttatgccgtggtttttgctgcattatcgatcctgctg | | |
| | | atttatctgggcagtacaggaatgtggccgctccagtggattggcgcaggtatgacggcgtatggattactcta | | |
| | | ttttatggtgcacgacgggctggtgcatcaacgttggcacttccgctatattccacgcaagggctacctcaaac | | |
| | | ggttgtatatggcgcaccgtatgcatcacgccgtcaggggcaaagaaggttgtgtttcttttggcttcctctat | | |
| | | gcgccgcccctgtcaaaacttcaggcgacgctccgggaaagacatggcgctagagcgggcgctgccagagatgc | | |
| | | gcagggcggggaggatgagcccgcatccgggaagGCCTAGAGACCACTAGTTGCGGCCGCTGCAG | | |
| | | (SEQ ID NO: 19) | | |
| VioA | | GAATTCGCGGCCGCTTCTAGAGGTCTCATGGGTcattcttccgatatctgcattgttggtgctggtatttctgg | K274002 | |
| | | tttgacgtgcgcaagccatctgctggacagcccggcatgccgtggtctgagcctgcgtatctttgacatgcagc | | |
| | | aagaagccggtggccgtatccgcagcaaaatgctggatggtaaggcaagcattgaactgggcgcaggtcgctac | | |
| | | tcccctcagttgcacccgcatttccaaagcgcaatgcagcactatagccaaaagagcgaagtctatccgttcac | | |
| | | ccagttgaagttcaaatctcacgtgcagcaaaagctgaagcgcgccatgaatgaactgtccccgcgtctgaaag | | |
| | | agcatggtaaagagagctttttgcagtttgtcagccgttatcaaggtcacgatagcgcggttggtatgatccgc | | |
| | | tctatgggttacgacgcactgttcctgccggatatcagcgcagaaatggcctacgacattgtgggtaagcaccc | | |
| | | ggagatccagagcgtgacggacaacgacgcgaaccaatggtttgcagcggaaacgggctttgctggtctgattc | | |
| | | agggcatcaaggctaaggttaaggcggcaggtgcgcgttttagcctgggttatcgtctgctgagcgtccgtacc | | |
| | | gacggtgacggctacctgctgcaactggcaggtgacgacggctggaaactggagcaccgtacccgccatctgat | | |
| | | tctggcgattccgccgagcgcgatggcgggtttgaatgttgattttccagaagcctggtccggtgcgcgctatg | | |
| | | gcagcctgccgctgtttaagggctttctgacgtacggtgagccgtggtggttggactacaaactggacgatcag | | |
| | | gtgctgattgttgacaacccgctgcgcaaaatctatttcaagcgaagtaagtacctgttcttctataccgatag | | |
| | | cgagatggcgaattactggcgcggttgtgtcgcggagggcgaggacggttacctggagcaaattcgcacccatt | | |
| | | tggctagcgcactgggtatcgtccgtgaacgtatcccgcaaccgctggcacacgttcacaagtattgggcgcac | | |
| | | ggcgttgagttttgccgtgattctgatattgaccacccgagcgcactgtctcatcgcgacagcggtatcatcgc | | |
| | | gtgctccgatgcgtacacggagcattgtggttggatggagggcggtctgctgagcgcccgtgaggcaagccgtc | | |
| | | tgctgttgcagcgtatcgccgcgGCCTAGAGACCACTAGTTGCGGCCGCTGCAG | | |
| | | (SEQ ID NO: 20) | | |
| VioB | | GAATTCGCGGCCGCTTCTAGAGGTCTCATGGGTattctggatttcccgcgtatccacttccgtggctgggcccg | K274002 | |
| | | tgtcaatgcgccgaccgcgaaccgcgatccgcacggccacatcgatatggccagcaataccgtggcgatggcgg | | |
| | | gtgagccgttcgacctggcacgccatcctacggagttccaccgtcacctgcgctccctgggtccgcgcttcggc | | |
| | | ttggatggtcgtgctgacccggaaggcccgttcagcctggccgagggctacaacgctgccggtaacaaccactt | | |
| | | ttcgtgggagagcgcaaccgttagccacgtgcaatgggatggcggtgaggcggatcgtggtgacggtctggtcg | | |
| | | gtgctcgtttggcactgtggggtcactacaatgattatctgcgcggtaccaccttcaatcgtgctcgttgggtc | | |
| | | gacagcgacccgacgcgccgtgacgctgcacaaatctatggccaattcaccattagcccggctggtgccggtcc | | |
| | | gggtacgccgtggctgtttacggcagacattgatgatagccatggtgcacgttggacgcgtggcggccacattg | | |
| | | cagagcgtggcggccacttcttggatgaagagtttggtctggcacgcctgtttcagttctctgtgccgaaagat | | |
| | | cacccacattttctgtttcacccgggtccgtttgattccgaggcctggcgtcgtctgcaattggctctggagga | | |
| | | tgacgacgttctgggtctgaccgtgcaatatgcgttgttcaatatgagcaccccgcctcagccgaacagcccgg | | |
| | | tttttcacgatatggtcggtgttgtcggtctgtggcgtcgtggtgaactggcgagctacccggctggtcgtctg | | |
| | | ctgcgtccgcgtcaaccgggtctgggtgacctgaccctgcgcgtcaacggtggtcgcgttgcgctgaatttggc | | |
| | | gtgtgccattccgttcagcactcgtgccgcgcagccaagcgcaccggaccgcctgaccccggacctgggtgcca | | |
| | | aactgccgctgggcgatctgctgctgcgtgatgaggacggcgcactgttggcacgtgtgccgcaggctctgtac | | |
| | | caagactattggacgaatcacggtattgtggacctgccgctgctgcgcgaaccgcgtggtagcttgaccctgag | | |
| | | cagcgaactggcggagtggcgtgagcaagactgggtcacccaaagcgacgcgtctaacctgtacctggaggcac | | |
| | | cggatcgccgtcacggtcgctttttccctgagagcatcgcgctgcgcagctactttcgcggtgaagcgcgtgcg | | |
| | | cgtccggatatcccgcatcgtatcgagggcatgggcctggtcggcgtcgaatctcgtcaggatggcgacgctgc | | |
| | | ggaatggcgtctgacgggtctgcgtccgggtccggcacgcattgttctggacgatggtgccgaggcgatccctc | | |
| | | tgcgtgttctgcctgacgattgggcgctggatgacgcgaccgtcgaagaagtggattacgcctttttgtaccgc | | |
| | | cacgttatggcgtattacgagctggtgtatccattcatgagcgacaaggtgttttccctggctgatcgttgcaa | | |
| | | atgtgaaacgtacgcacgtctgatgtggcagatgtgtgatccgcagaaccgcaacaagtcctattacatgccga | | |
| | | gcacccgcgaactgtcggcaccgaaagctcgtttgttcttgaagtatctggcccacgtggaaggccaggcacgc | | |
| | | ctgcaagcacctccgccagcgggtccggcacgcattgaatctaaagcccagttggcggcagagctgcgtaaagc | | |
| | | cgtcgacctggagctgtctgtgatgctgcaatacctgtacgcggcgtatagcattccgaactatgcacagggcc | | |
| | | aacaacgtgttcgtgacggtgcgtggaccgccgagcagctgcaactggcgtgcggtagcggtgaccgtcgccgt | | |
| | | gatggcggtattcgtgcagcactgctggaaattgctcatgaagaaatgattcattacctggtcgttaacaacct | | |
| | | gctggatgccctgggcgagccgttctacgcgggtgtcccgctgatgggcgaagcggcacgtcaggcgtttggcc | | |
| | | tggacaccgagttcgctctggaaccgtttagcgaaagcacgctggcacgttttgttcgtctggaatggccgcac | | |
| | | tttatcccagcaccgggcaaatccatcgcggactgctatgccgccattcgtcaggcgtttttggatctgccgga | | |
| | | cttgtttggtggcgaggcaggtaagcgtggcggtgaacaccacctgttcctgaatgagctgaccaaccgtgcgc | | |
| | | atccgggttatcaactggaagttttcgatcgcgactcggcgctgtttggtattgcatttgtgaccgatcagggc | | |
| | | gaaggtggcgctctggacagcccgcactacgaacatagccattttcaacgtctgcgtgaaatgagcgcgcgtat | | |
| | | catggctcaaagcgcaccgttcgaaccggcgctgccggcgttgcgtaatccggttctggatgagagcccgggtt | | |
| | | gccaacgtgtcgcagacggtcgtgcgcgtgcgctgatggcattgtaccaaggcgtttatgagctgatgtttgcg | | |
| | | atgatggcgcagcacttcgccgtgaaaccgctgggtagcttgcgtcgcagccgcctgatgaacgcagcaatcga | | |
| | | atctgatgaccggtctgttgcgtccgctgagctgcgcgctgatgaacctgccaagcggcatcgccggtcgccgg | | |
| | | ccggtccgccgctgccgggtccggttgacacccgtagctatgacgactacgcgctgggctgtcgcatgctggca | | |
| | | cgccgttgcgagcgtctgctggagcaggcgagcatgctggaaccgggttggctgccggatgcgcagatggagct | | |
| | | gctggatttctatcgtcgccaaatgctggacttggcgtgcggcaaactgagccgcgaggccGCCTAGAGACCAC | | |
| | | TAGTTGCGGCCGCTGCAG | | |
| | | (SEQ ID NO: 21) | | |
| VioC | | GAATTCGCGGCCGCTTCTAGAGGTCTCATGGGTcgtgcgattatcgttggtggcggcctggcgggtggcctgac | K274002 | |
| | | cgcgatctacctggcgaagcgtggctacgaagtgcacgtcgtggagaagcgtggtgatcctctgcgcgatctga | | |
| | | gctcttacgtggacgttgttagcagccgtgcgatcggcgtgagcatgaccgttcgtggtatcaagagcgttttg | | |
| | | gctgcgggcattccgcgtgcagagctggatgcgtgtggcgaaccgatcgtggcaatggctttctccgtgggtgg | | |
| | | tcagtatcgcatgcgcgaactgaagccgttggaggatttccgtccgctgagcttgaaccgtgcggcgtttcaaa | | |
| | | agctgctgaacaaatacgcgaacctggcaggcgttcgttactactttgagcataagtgcctggatgttgacctg | | |
| | | gatggtaagagcgtgttgattcagggcaaagatggtcagccgcagcgtctgcaaggtgacatgattatcggtgc | | |
| | | ggatggcgcccacagcgccgtccgtcaggcgatgcagagcggcctgcgtcgtttcgagttccagcaaacgttct | | |
| | | tccgccatggctacaaaaccctggttttgccggacgcgcaagcactgggttaccgtaaagacacgctgtacttt | | |
| | | ttcggcatggattccggtggcctgttcgcgggtcgtgcggctacgatcccagatggtagcgtcagcatcgccgt | | |
| | | ttgcctgccgtactcgggtagcccttccctgacgaccaccgacgaaccgacgatgcgtgcgttcttcgatcgtt | | |
| | | acttcggtggcctgccgcgtgacgcgcgtgacgaaatgctgcgtcagtttctggcgaagccgagcaacgacctg | | |
| | | attaacgtgcgctctagcacctttcactataagggtaatgtgctgttgctgggtgatgctgcgcatgcgactgc | | |
| | | gccgttcctgggtcagggtatgaacatggcgctggaggacgcccgcacgtttgtcgagctgctggaccgccacc | | |
| | | agggcgaccaagacaaagcctttccggagttcacggagctgcgcaaagtccaggcagacgcaatgcaagacatg | | |
| | | gctcgcgccaactatgacgttttgagctgctcgaacccgatctttttcatgcgtgcgcgttacacgcgttacat | | |
| | | gcattccaagtttccgggcctgtatccgccggatatggccgagaaactgtactttacgagcgagccgtacgatc | | |
| | | gtctgcaacaaatccagcgtaaacagaatgtttggtacaagattggtcgcgtgGCCTAGAGACCACTAGTTGCG | | |
| | | GCCGCTGCAG | | |
| | | (SEQ ID NO: 22) | | |
| VioD | | GAATTCGCGGCCGCTTCTAGAGGTCTCATGGGTattctggtcattggtgctggtccagctggtctggttttcgc | K274002 | |
| | | atcccaactgaagcaggcacgccctttgtgggccattgacatcgtggagaagaatgacgagcaagaagtgctgg | | |
| | | gctggggtgtcgtgctgcctggccgtccgggtcagcacccggcgaacccgctgtcctatctggatgcaccggag | | |
| | | cgtctgaatccgcaatttctggaggacttcaaactggtgcatcataatgagccgtccttgatgtccacgggcgt | | |
| | | tttgttgtgcggcgtggagcgtcgcggtctggttcacgcgctgcgcgataagtgccgcagccaaggcattgcta | | |
| | | ttcgtttcgaaagcccgttgctggaacacggtgagctgccgctggcggactatgatctggtggtcctggctaat | | |
| | | ggtgttaatcacaaaaccgcgcatttcaccgaggctctggtcccgcaggtggactacggccgcaataagtacat | | |
| | | ttggtatggcactagccagctgttcgatcagatgaatctggtttttcgtacccatggtaaagatatctttatcg | | |
| | | cgcatgcctataagtatagcgataccatgagcacgttcattgtcgaatgtagcgaagagacttacgcacgcgca | | |
| | | cgcctgggcgaaatgtccgaagaggcgagcgcagaatacgttgctaaggtgttccaggccgagctgggtggtca | | |
| | | cggcctggtgagccagccgggtctgggttggcgtaacttcatgacgttgtctcatgaccgttgtcatgatggta | | |
| | | agttggttctgctgggtgacgcgctgcaaagcggtcactttagcatcggccacggcaccacgatggccgtggtg | | |
| | | gtggcgcagctgctggttaaagcgctgtgtaccgaagatggtgtgcctgccgcgctgaaacgtttcgaagagcg | | |
| | | tgccctgccgctggtgcagttgttccgtggccacgcagacaacagccgcgtttggttcgaaaccgtcgaagagc | | |
| | | gcatgcacctgtcctcggcggaatttgtgcaaagcttcgacgcacgccgcaaagcctgccgccgatgccggaag | | |
| | | cactggcgcagaatctgcgttatgcttatgcagGCCTAGAGACCACTAGTTGCGGCCGCTGCAG | | |
| | | (SEQ ID NO: 23) | | |
| VioE | | GAATTCGCGGCCGCTTCTAGAGGTCTCATGGGTaaccgtgagccaccactgttgccagcccgttggagcagcgc | K274002 | |
| | | ctatgtctcttattggagcccgatgctgccggatgaccagctgaccagcggctattgctggttcgactatgaac | | |
| | | gtgacatctgtcgtattgacggcctgttcaatccgtggagcgagcgtgatactggttatcgcctgtggatgtcg | | |
| | | gaggttggtaatgcggccagcggccgtacctggaaacaaaaagtcgcctatggtcgtgagcgtaccgccctggg | | |
| | | tgaacagctgtgtgagcgtccgctggatgatgagactggcccttttgccgaattgttcctgccacgcgatgtcc | | |
| | | tgcgccgtctgggtgcccgtcacattggccgtcgcgtggttctgggtcgcgaagcggacggttggcgttaccag | | |
| | | cgcccaggtaaaggtccgagcaccctgtacctggatgcggcgagcggcactccactgcgcatggtcaccggcga | | |
| | | tgaagcgtcgcgtgcaagcctgcgtgattttccgaatgtgagcgaggcggagatcccggacgcggttttcgcgg | | |
| | | ccaagGCCTAGAGACCACTAGTTGCGGCCGCTGCAG | | |
| | | (SEQ ID NO: 24) | | |
| Pr1000 | BBy_ | GCCTttgacggctagctcagtcctaggtacagtgctagcAAGTTCACGTAGGAGGACAGCTATGGG | J23100 | added |
| | 106 | (SEQ ID NO: 25) | se- | Trp |
| | | | quence, | leader |
| | | | syn- | and |
| | | | thetic | RBS, |
| | | | | con- |
| | | | | verted |
| | | | | to Byte |
| | | | | format |
| Pr562 | BBy_ | GCCTtttacggctagctcagtcctaggtatagtgctagcAAGTTCACGTAGGAGGACAGCTATGGG | J23106 | added |
| | 107 | (SEQ ID NO: 26) | pro- | Trp |
| | | | moter, | leader |
| | | | syn- | and |
| | | | thetic | RBS, |
| | | | | con- |
| | | | | verted |
| | | | | to Byte |
| | | | | format |
| Pr248 | BBy_ | GCCTtttacggctagctcagtcctaggtactatgctagcAAGTTCACGTAGGAGGACAGCTATGGG | J23105 | added |
| | 108 | (SEQ ID NO: 27) | se- | Trp |
| | | | quence, | leader |
| | | | syn- | and |
| | | | thetic | RBS, |
| | | | | con- |
| | | | | verted |
| | | | | to Byte |
| | | | | format |
| Pr150 | BBy_ | GCCTtttatggctagctcagtcctaggtacaatgctagcAAGTTCACGTAGGAGGACAGCTATGGG | J23114 | added |
| | 109 | (SEQ ID NO: 28) | se- | Trp |
| | | | quence | leader |
| | | | | and |
| | | | | RBS, |
| | | | | con- |
| | | | | verted |
| | | | | to Byte |
| | | | | format |
| Pr64 | BBy_ | GCCTtttacagctagctcagtcctagggactgtgctagcAAGTTCACGTAGGAGGACAGCTATGGG | J23109 | added |
| | 110 | (SEQ ID NO: 29) | | Trp |
| | | | | leader |
| | | | | and |
| | | | | RBS, |
| | | | | con- |
| | | | | verted |
| | | | | to Byte |
| | | | | format |
| PrAraC | BBy_ | tagcaagatagtccataagattagcggatcctacctgacgctttttatcgcaactctctActgtttctccata | BBa_ | |
| | 114 | (SEQ ID NO: 30) | K206000 | |
| Pr | BBy_ | GCCTTATCCCTTGCGGTGATAGATATTTATCCCTTGCGGTGATAGATTTAACGTAAGTTCACGTA | | |
| lambda | 113 | GGAGGACAGCTATGGG | | |
| C1 | | (SEQ ID NO: 31) | | |
| PrLacI | BBy_ | GCCTATAAATGTGAGCGGATAACATTGACTTGTGAGCGGATAACAAGATACTGAGCACAAGTT | | |
| | 111 | CACGTAGGAGGACAGCTATGGG | | |
| | | (SEQ ID NO: 32) | | |
| PrTetRo | BBy_ | GCCTTCCCTATCAGTGATAGAGATTGACTCCCTATCAGTGATAGAGATACTGAGCACAAGTTCA | | |
| | 112 | CGTAGGAGGACAGCTATGGG | | |
| | | (SEQ ID NO: 33) | | |
| term | BBy_ | gcctaataaagatacccagcccgcctaatgagcgggcttttttttTGGG | | |
| TrpAttr | 103 | (SEQ ID NO: 34) | | |
| termNeg | BBy_ | gcctaataaagatacccgcgggctctaatgagcgggcttttttttTGGG | | |
| Control | 104 | (SEQ ID NO: 35) | | |
| linkStp | | native sequence | | |
| Rbs | | (SEQ ID NO: 36) | | |
| linkNeg | | native sequence | | |
| Control | | (SEQ ID NO: 37) | | |
| End | BBy_ | TGGGAACCTCCTCTCGAAGCCT | | |
| In- | 105 | (SEQ ID NO: 38) | | |
| verter | | | | |
| AtoB | | | | |
| End | BBy_ | GCCTAACCTCCTCTCGAATGGG | | |
| In- | 102 | (SEQ ID NO: 39) | | |
| verter | | | | |
| BtoA | | | | |
| link | BBy_ | GCCTAAGGAGGACAGCTATGGG | | |
| Operon | 101 | (SEQ ID NO: 40) | | |

### Example 6

### Cap-anchor interaction facilitates ligation-free transformation

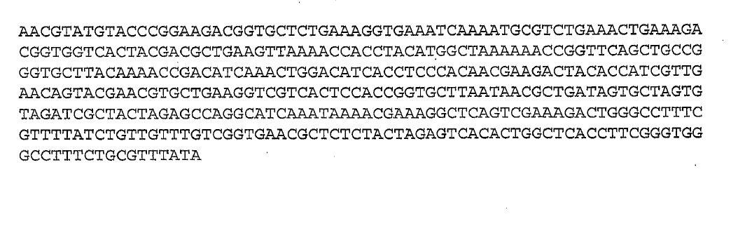

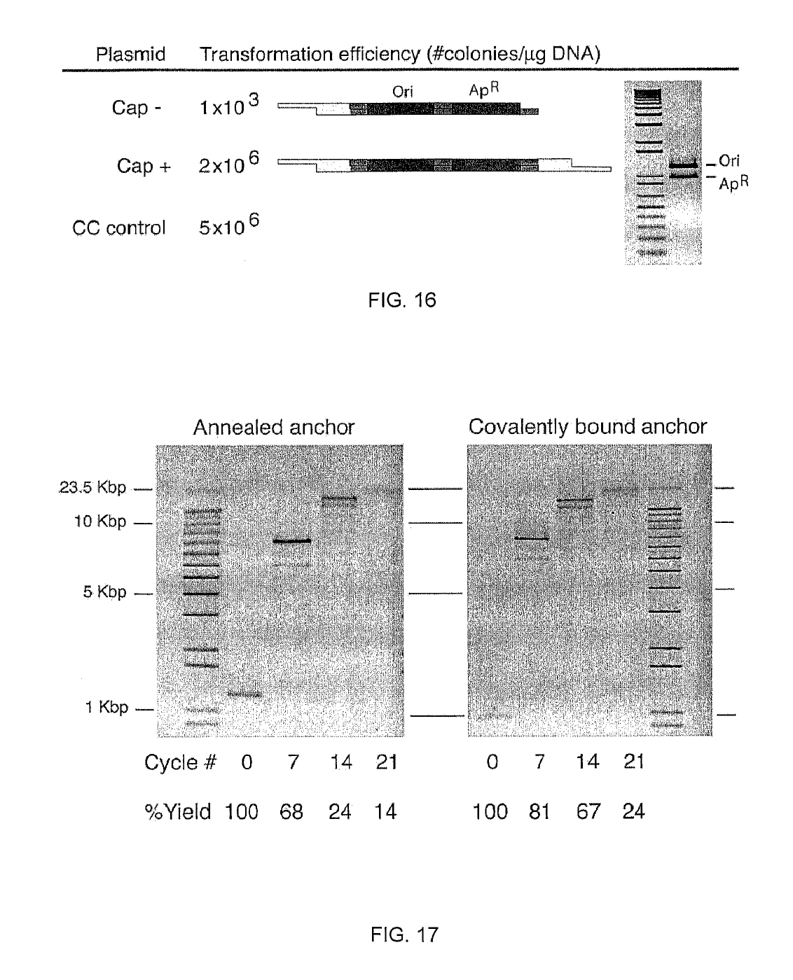

As shown in FIG. 16, a three-part assembly (Cap +), consisting of an origin of replication (Ori) bound to an anchor sequence, an ampicillin resistance cassette (Ap^{R}), and a terminal cap, was assembled as described, except with elution from the bead being accomplished using 20 ml of 10 mM NaOH instead of elevated temperature, followed with neutralization by adding 2 μl of 0.5 M Tris. As a negative control (Cap −), a construct without the cap but otherwise identical was constructed. The eluted product was transformed into chemically competent DH5α E. coli cells, and transformation efficiencies were computed. The cap and anchor function together to produce a highly transformable circular plasmid, with a transformation efficiency approaching that of the supercoiled positive control (CC control). Of the cap positive transformants, 342 colonies were collected and pooled, plasmid DNA isolated from the pooled collection, cut with a restriction enzyme, and run on a gel. The absence of variant bands indicates that all or essentially all of the colonies have the sequence of the desired construct.

### Example 7

### Assembly of a 23 kb, 22 Part Construct

To an anchoring fragment of 1.2 kb, 21 successive additions added alternately a 1 kb AB piece and a short (<100 bp) BA linker, removing a fraction for analysis every seven cycles. All were eluted and run on a gel. An assembly was also constructed with an anchoring sequence designed to bind covalently to the bead, and released by digestion with a restriction enzyme whose recognition sequence was designed into the anchor. As shown in FIG. 17, the lanes represent: ladder, anchor, intermediate after 7 steps, 14 steps, and the final product after 21 steps; left: annealed anchor, right: covalently bound anchor. Molar yields were computed after band densitometry, corrected for bead loss. The average coupling efficiency per step over 21 steps was 91% for the annealed anchor, and 93% for the covalently bound anchor. The coupling efficiency for up to 14 steps with a covalently bound anchor was more than 97%. While some incomplete constructs are visible, the majority product remains the full length construct even at the largest size.

### Example 8

### Assembly from a Gene Synthesized from Ligated Oligonucleotides

This example demonstrates both the efficiency and precision of the assembly method of the present invention, and the utility of ligation gene synthesis for production of linear DNA with the desired overhangs. Parts with desired overhangs were produced by direct ligation of synthetic oligonucleotides. The lacZα fragment from pUC19 was divided into segments of about 30 bp, with 4 by overhangs, and synthesized as a set of oligonucleotides. With promoter, terminator, and proximal and distal overhangs, it constituted 358 bp. After annealing and one-pot ligation, gel analysis showed that less than 30% was full length product. The entire ligated mixture was used as an AB part in sequential assembly with an origin and resistance marker, transformed, and 20 of the resulting colonies were selected for sequencing. Of these colonies, 100% included the synthetic insert, demonstrating that the assembly method is highly selective for the correct product. 13/20 of these colonies were in 100% agreement with the designed sequence, with the mutations consisting of deletions, insertions, and mismatches resulting from oligonucleotide synthesis occurring at an overall rate of 1.3 errors/kb. A separate assembly and transformation again resulted in 100% assembly across 12 sequenced colonies, with 5 synthesis mutations. The locations of the mutations from both runs are shown in FIG. 18.

### Example 9

### Constructing overhangs by heteroduplexing linear PCR products

By heteroduplexing a pair of linear PCR products, each of which incorporates one end but not the other, the desired overhang product can be easily made (Tillett et al., 1999; Matsumoto et al., 2011). This method was successful in generating the product shown in FIGS. 19A-B. This method is also useful in generating long tailed ends, such as those required for anchors and caps. While the nominal yield after annealing is only 25% of the starting material, this can be increased through methods such as annealing in the presence of a selective complementary end, such as provided by the oligo-dT beads, or by producing an excess of the desired single strand in the PCR product, through asymmetric or entirely single-sided PCR (Sanchez et al., 2004). Other methods of selective purification of single-stranded DNA may also be useful (Kuo, 2005). A high-fidelity polymerase is required to leave precise 3′ ends, without added untemplated bases. Polymerases such as Taq which add a terminal untemplated adenosine can be accommodated with small changes in end and part sequences.

### Example 10

### Buffer Optimization for Spin Column Purification

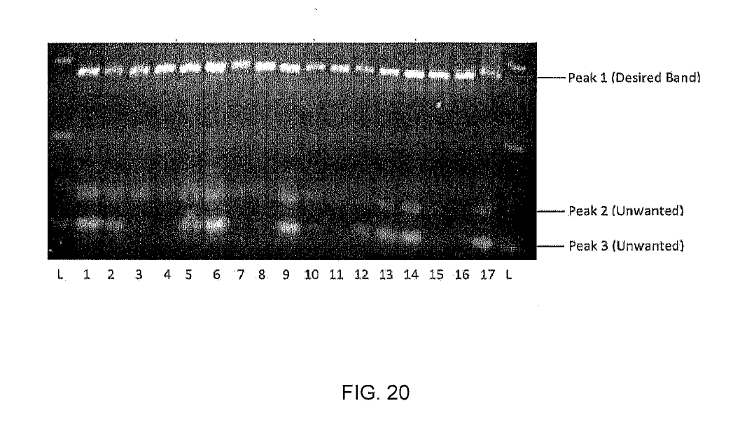

Fluorescently labelled primers in combination with gel electrophoresis may be used to indicate what fraction of a PCR fragment has been successfully cleaved by a restriction enzyme, and what fraction of the cleaved ends have been cleaned away from the preparation by the separation procedure. FIG. 20 illustrates the use of fluorescently labelled primers in a study of binding buffer conditions in spin column separation of DNA from small ends. A linear PCR product with 5-FAM labelled ends 30 bp from a BsaI site were digested, bound to a silica spin column using a variety of buffers, eluted and visualized after gel electrophoresis. Binding buffers analyzed were: Qiagen PB (Qiagen Inc., Toronto, ON) (lanes 1, 5, 9, 13); 5.5 M GuHCl, 20 mM Tris-HCl (lanes 2, 6, 10, 14); 7 M GuHCl, 5 mM KCl, 1 mM Tris-HCl, 0.15 mM MgCl₂, 0.01% Triton™ X-100 pH 5.5 (based on Padhye, 1998, lanes 3, 7, 11, 15); 5 M GuHCl, 10 mM Tris HCl, 30% ethanol, pH 6.6 (based on Anonymous, 2011; lanes 4, 8, 12, 16). Wash buffers analyzed were: Qiagen PE (Qiagen Inc., Toronto, ON) (lanes 1-4); 5 mM NaCl, 2 mM Tris HCl, 80% ethanol, pH 7.5 (lanes 5-8); 83 mM NaCl, 8.3 mM Tris HCl, 2.1 mM EDTA, 55% ethanol pH 7.5 (based on Padhye, 1998; lanes 9-12); and 10 mM Tris HCl, 80% ethanol, pH 7.5 (Anonymous, 2011; lanes 13-16). Lane 17 holds an unseparated control. The best combination is in lane 16, which provides a superior separation of the desired product from the contaminant than standard Qiagen™ buffers or other buffers known in the art.

### REFERENCES

The following references are incorporated herein by reference (where permitted) as if reproduced in their entirety. All references are indicative of the level of skill of those skilled in the art to which this invention pertains.
Anderson, J. C., Dueber, J. E., Leguia, M., Wu, G. C., Goler, J. A., Arkin, A. P. and Keasling, J. D. (2010) BglBricks; a flexible standard for biological part assembly. J. Biol. Eng. 4:1.
Anonymous. Qiagen Buffers—OpenWetWare [http://openwetware.org/wiki/Qiagen_Buffers]. Accessed Aug. 17, 2011.
Arkin, A. (2008) Setting the standards in synthetic biology. Nat. Biotechnol. 26(7):771-774.
Beattie, K. L. and Fowler, R. F. (1991) Solid-phase gene assembly. Nature 352(6335):548-549.
Bitinaite, J., Rubino, M., Varma, K. H., Schildkraut, I., Vaisvila, R. and Vaiskunaite, R. (2007) USER™ friendly DNA engineering and cloning method by uracil excision. Nucleic Acids Res. 35(6):1992-2002.
Carr, P. A. and Church, G. M. (2009) Genome engineering. Nat. Biotechnol. 27:1151-1162.
Church, G. and Pitcher, E. Accessible polynucleotide libraries and methods of use thereof. United States Patent Application Publication No. 2006/0281113, published Dec. 14, 2006.
Colpan, M., Schorr, J., Herrmann, R. and Feuser, P. Chromatographic purification and separation process for mixtures of nucleic acids. U.S. Pat. No. 6,383,393, issued May 7, 2002.
Dietrich, G., Bubert, A., Gentschev, I., Sokolovic, Z., Simm, A., Catic, A., Kaufmann, S. H., Hess, J., Szalay, A. A. and Goebel, W. (1998) Delivery of antigen-encoding plasmid DNA into the cytosol of macrophages by attenuated suicide Listeria monocytogenes. Nat. Biotechnol. 16(2):181-185.
Endy, D. (2005) Foundations for engineering biology. Nature 438:449-453.
Ellis, T., Adie, T., and Baldwin, G. S. (2011) DNA assembly for synthetic biology: from parts to pathways and beyond. Integr. Biol. 3:109-118.
Gibson, D. G. and Young, L. Assembly of large nucleic acids. United States Patent Application Publication No. 2009/275086, published Nov. 5, 2009.
Hartley, J. L., Temple, G. F. and Brasch, M. A. (2000) DNA cloning using in vitro site-specific recombination. Genome Research 10:1788-1795.
Harvey, P. D. Method and kits for preparing multicomponent nucleic acid constructs. U.S. Pat. No. 6,277,632, issued Dec. 17, 2002.
Heckman, K. L. and Pease, I. R. (2007) Gene splicing and mutagenesis by PCR-driven overlap extension. Nature Protocols 2:924-932.
Hostomsky, Z., Smrt, J., Arnold, L., Tocik, Z. and Paces, V. (1987a) Solid-phase assembly of cow colostrum trypsin inhibitor gene. Nucleic Acids Res. 15(12):4849-4856.
Hostomsky, Z. and Smrt, J. (1987b) Solid-phase assembly of DNA duplexes from synthetic oligonucleotides. Nucleic Acids Symp Ser 18:241-244.
Jarrell, K. A. and Coljee, V. W. Ordered gene assembly. U.S. Pat. No. 6,358,712, issued Mar. 19, 2002.
Kuo, T. C. (2005) Streamlined method for purifying single-stranded DNA from PCR products for frequent or high-throughput needs. Biotechniques 38(5):700, 702.
Li, M. and Elledge, S. J. (2007) Harnessing homologous recombination in vitro to generate recombinant DNA via SLIC. Nature Methods 4:251-256.
Matsumoto, A. and Itoh, T. Q. (2011) Self-assembly cloning: a rapid construction method for recombinant molecules from multiple fragments. Biotechniques 51(1):55-56.
Mulligan, J. T. and Tabone, J. C. Methods for improving the sequence fidelity of synthetic double-stranded oligonucleotides. U.S. Pat. No. 6,664,112, issued Dec. 16, 2003.
Mulligan, J. T., Tabone, J. C. and Brickner, R. G. Method and system for polynucleotide synthesis. U.S. Pat. No. 7,164,992, issued Jan. 16, 2007.
Padhye, V. V., York, C. and Burkiewicz, A. Nucleic acid purification using silica gel and glass particles. U.S. Pat. No. 5,808,041, issued Sep. 15, 1998.
Parker, H. Y. and Mulligan, J. T. (2003) Solid phase methods for polynucleotide production. United States Patent Application Publication No. 2003/0228602 A1, published Dec. 11, 2003.
Parker, H. Y. and Mulligan, J. T. (2009) Solid phase methods for polynucleotide production. U.S. Pat. No. 7,482,119, issued Jan. 27, 2009.
Registry of Standard Biological Parts. http://www.partsregistry.org.
Sanchez, J. A., Pierce, K. E., Rice, J. E. and Wangh, L. J. (2004) Linear-after-the-exponential (LATE)-PCR: an advanced method of asymmetric PCR and its uses in quantitative real-time analysis. Proc Natl Sci USA 101(7):1933-1938.
Shetty, R. P., Endy, D. and Knight, T. F. J. (2008) Engineering BioBrick vectors from BioBrick parts. J. Biol. Eng. 2:5.
Sleight, S. C., Bartley, B. A., Lieviant, J. A. and Sauro, H. M. (2010) In-Fusion bioBrick assembly and re-engineering. Nucleic Acids Res. 38(8):2624-2636.
Tillett, D. and Neilan, B. A. (1999) Enzyme-free cloning: a rapid method to clone PCR products independent of vector restriction enzyme sites. Nucleic Acids Res. 27(19):e26-e28.
Xiong, A. S, Peng, R. H., Zhuang, J., Liu, J. G., Gao, F., Chen, J. M., Cheng, Z. M. and Yao, Q. H. (2008) Non-polymerase-cycling-assembly-based chemical gene synthesis: strategies, methods, and progress. Biotechnol Adv. 26(2):121-134.

What is claimed is:

1. A method for assembly of a DNA construct comprising the steps of:
a) incubating a support with a first form of nucleic acid components under conditions to form support-bound nucleic acid component complexes;
b) removing unbound first form nucleic acid components;
c) incubating the support-bound first form nucleic acid component complexes with a second form of nucleic acid components under conditions to anneal and link the second form to the first form;
d) removing unbound second form nucleic acid components;
e) repeating steps c) and d) until the DNA construct is generated; and
f) eluting the DNA construct from the support;
wherein the first and second form of nucleic acid component comprises sticky ends such that each form cannot link to itself but can link to each other to form an alternating head to tail sequence.

2. The method of claim 1, wherein each sticky end is nonpalindromic.

3. The method of claim 2, wherein each sticky end comprises a sequence within a predetermined set of sequences.

4. The method of claim 3, wherein each sticky end comprises a sequence as set forth in any one of SEQ ID NOS: 53 to 71.

5. The method of claim 3, wherein two sticky ends comprise SEQ ID NOS: 53 and 54 respectively.

6. The method of claim 3, wherein two sticky ends comprise SEQ ID NOS: 55 and 56 respectively.

7. The method of claim 3, wherein a nucleic acid component comprises SEQ ID NO: 53 at one end, and SEQ ID NO: 56 at the other end.

8. The method of claim 7, wherein the nucleic acid component comprises SEQ ID NO: 55 at one end, and SEQ ID NO: 54 at the other end.

9. The method of claim 2, wherein the sticky end has a length of about 4 base pairs.

10. The method of claim 1, wherein a nucleic acid component comprises one or more nucleic acid sequences providing one or more biological functionalities.

11. The method of claim 10, wherein the one or more biological functionalities comprises origin of replication, selectable marker, transcriptional regulatory element, structural gene or fragment thereof, transcription termination signal, translational regulatory sequence, regulators of mRNA stability, cellular localization signal, recombination elements, mutagenized genes, protein domain encoded regions, synthetic multiple cloning sites, unique restriction enzyme or DNA cleavage sites, and site for covalent or non covalent attachment of a biological or chemical molecule.

12. The method of claim 10, wherein the nucleic acid sequence provides an open reading frame lacking initiation and termination codons.

13. The method of claim 10, wherein the nucleic acid sequence provides a ribosome binding site, initiation and termination codons, and a linker for an open reading frame.

14. The method of claim 1, wherein a nucleic acid component comprises a sequence as set forth in any one of SEQ ID NOS: 1 to 40.

15. The method of claim 1, wherein a nucleic acid component comprises an anchor sequence annealed or covalently bound to the support.

16. The method of claim 15, wherein the anchor sequence comprises a 5′ sticky poly-dA, a Type IIs restriction site, and a 3′ terminal sequence.

17. The method of claim 16, wherein the 3′ terminal sequence comprises a sequence selected from 5′-TGGG or 5′-GCCT.

18. The method of claim 15, wherein the support comprises a bead or microsphere capable of binding the anchor sequence.

19. The method of claim 1, wherein a nucleic acid component comprises a terminator sequence comprising a poly-dT end cap.

20. The method of claim 1, wherein a nucleic acid component comprises a direction reversing linker.

21. The method of claim 1, wherein the nucleic acid components are incubated in a step-wise manner.

22. The method of claim 1, wherein the nucleic acid components are incubated simultaneously.

23. The method of claim 1, wherein the elution of step (f) comprises treatment with heat, an elution buffer, or both.

24. The method of claim 23, wherein the elution buffer comprises a sodium hydroxide solution.

25. The method of claim 1, further comprising transforming a host cell with the eluted DNA construct.

26. The method of claim 25, wherein the host cell comprises an E. coli cell.

27. The method of claim 1, wherein the DNA construct comprises a size greater than 1 kb.

28. A kit for assembly of a DNA construct comprising a plurality of first form and second form nucleic acid components, each nucleic acid component comprising double-stranded DNA having sticky ends to allow for annealing and linking of the nucleic acid components in a predetermined order, wherein the first and second form of nucleic acid component comprises sticky ends such that each form cannot link to itself but can link to each other to form an alternating head to tail sequence

29. The kit of claim 28, comprising a sequence as set forth in any one of SEQ ID NOS: 1-40 and 45-50.

30. A composition comprising one or more nucleic acid components as set forth in any one of SEQ ID NOS: 1-40 and 45-50.

31. A vector comprising a sequence as set forth in any one of SEQ ID NOS: 45-50.
